(19) United States
(12) Patent Application Publication
(10) Pub. No.: US 2024/0083844 A1
(43) Pub. Date: Mar. 14, 2024

(54) KINASE INHIBITORS AND USES THEREOF

(71) Applicant: ABM Therapeutics Corporation, San Diego, CA (US)

(72) Inventor: Chen CHEN, San Diego, CA (US)

(73) Assignee: ABM Therapeutics Corporation, San Diego, CA (US)

(21) Appl. No.: 18/266,793

(22) PCT Filed: Dec. 16, 2021
(86) PCT No.: PCT/US2021/063724

§ 371 Date: Jun. 12, 2023

Related U.S. Application Data
(60) Provisional application No. 63/126,364, filed on Dec. 16, 2020.

(51) Int. Cl. C07D 205/04; A61P 35/00; C07D 207/14; C07D 211/56; C07D 401/12

(52) U.S. Cl. CPC C07D 205/04; A61P 35/00; C07D 207/14; C07D 211/56; C07D 401/12

(57) ABSTRACT

Provided are kinases inhibitors, pharmaceutical compositions comprising such compounds, and methods of using such compounds or compositions, such as methods of treating a proliferation disorder, such as a cancer or a tumor, or in some embodiments disease or disorders related to the dysregulation of kinase such as, but not limited to kinases such as MEK, COT1, FGFR4, MINK, MYO3A, PKG1B, and PLK3.

### CROSS-REFERENCE TO RELATED APPLICATION

This application claims priority to and the benefit of U.S. Provisional Application No. 63/126,364, filed on Dec. 16, 2020, the disclosure of which is incorporated herein by reference in its entirety.

### FIELD

The present disclosure relates to compounds, pharmaceutical compositions comprising such compounds, and use of such compounds or compositions in methods of treatment or in medicaments for treatment of a proliferation disorder, a cancer or a tumor, or in some embodiments diseases or disorders related to the dysregulation of kinase such as, but not limited to MEK kinase.

### BACKGROUND

The present disclosure relates to the treatment of abnormal cell growth in mammals especially humans, such as cancer and, more specifically solid tumors and brain tumors, with novel cyclic amines described therein, and their isotopic derivatives as well as pharmaceutical compositions containing such compounds. In addition, the present disclosure relates to the methods of preparing such compounds.

A kinase is an enzyme that catalyzes the transfer of phosphate groups from high-energy, phosphate-donating molecules to specific substrates. This process is known as phosphorylation, where the substrate gains a phosphate group and the high-energy ATP molecule donates a phosphate group. This transesterification produces a phosphorylated substrate and ADP.

Kinases are classified into broad groups by the substrate they act upon: protein kinases, lipid kinases, carbohydrate kinases. Kinases can be found in a variety of species, from bacteria to mold to worms to mammals. More than five hundred different kinases have been identified in humans.

MAP kinases (MAPKs) are a family of serine/threonine kinases that respond to a variety of extracellular growth signals. For example, growth hormone, epidermal growth factor, platelet-derived growth factor, and insulin are all considered mitogenic stimuli that can engage the MAPK pathway. Activation of this pathway at the level of the receptor initiates a signaling cascade whereby the Ras GTPase exchanges GDP for GTP. Next, Ras activates Raf kinase (also known as MAPKKK), which activates MEK (MAPKK). MEK activates MAPK (also known as ERK), which can go on to

regulate transcription and translation. Whereas RAF and MAPK are both serine/threonine kinases, MAPKK is a tyrosine/threonine kinase.

The carcinogenic potential of the MAPK pathway makes it clinically significant. It is implicated in cell processes that can lead to uncontrolled growth and subsequent tumor formation. Mutations within this pathway alter its regulatory effects on cell differentiation, proliferation, survival, and apoptosis, all of which are implicated in various forms of cancer.

It is known that such kinases are frequently aberrantly expressed in common human cancers such as melanoma, colorectal cancer, thyroid cancer, glioma, breast cancer and lung cancer.

Inhibition of kinases is a useful method for disrupting the growth of mammalian cancer cells, therefore, for treating certain forms of cancer. Various compounds, such as pyrrolopyridine and anilinopyrimidine derivatives, have been shown to possess kinase inhibitory properties. Many patent publications refer to certain bicyclic derivatives, in particular quinazolinone derivatives.

Several compounds with diversified chemical structures have been developed into MEK inhibitors, and four of them (Trametinib, cobimetinib, binimetinib and selumetinib) are described as potent allosteric MEK1/2 inhibitors. For example, Trametinib inhibits the MEK1/2 enzyme at a low nanomolar range.

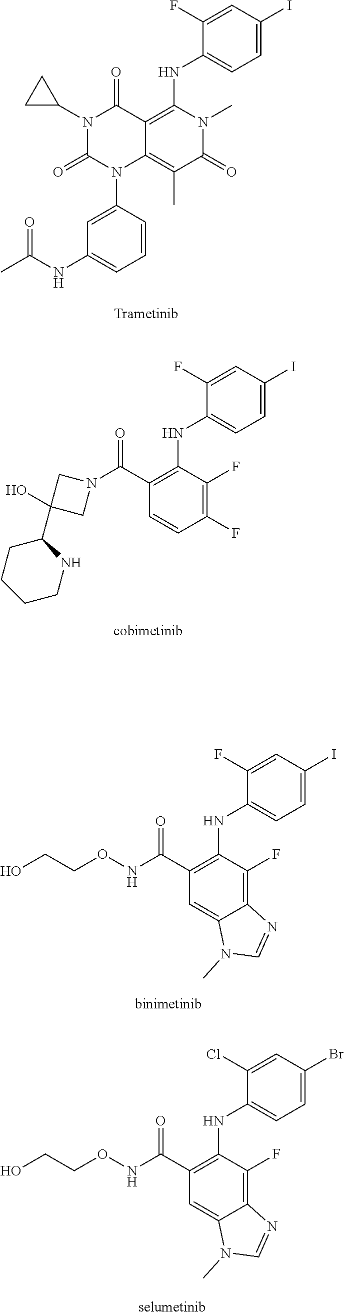 [SMILES: CC(=O)Nc1cccc(-n2c(=O)n(C3CC3)c(=O)c3c(Nc4ccc(I)cc4F)n(C)c(=O)c(C)c32)c1 ; O=C(c1ccc(F)c(F)c1Nc1ccc(I)cc1F)N1CC(O)([C@@H]2CCCCN2)C1 ; Cn1cnc2c(F)c(Nc3ccc(I)cc3F)c(C(=O)NOCCO)cc21 ; Cn1cnc2c(F)c(Nc3ccc(Br)cc3Cl)c(C(=O)NOCCO)cc21]

However, due to their structural characteristics, many of these kinase inhibitors exhibit poor pharmacokinetical properties, and some of them are substrates of active transporters such as P-glycoproteins (P-gp) or breast cancer resistance protein (BCRP), and have very low tendency to penetrate into cell membrane, as well as into brain. Therefore, they are not suitable to be used for the treatment of tumors or cancers in the brain, which is protected by the blood-brain barrier (BBB).

Thus, the compounds of the present disclosure, which are selective inhibitors of certain kinases, are useful in the treatment of abnormal cell growth, in particular cancers in mammals. In addition, these compounds have good penetration of cell membrane, therefore, are useful for treating tumors or cancers, including brain tumors, in humans.

### SUMMARY

In one aspect, provided is a compound of Formula (I):

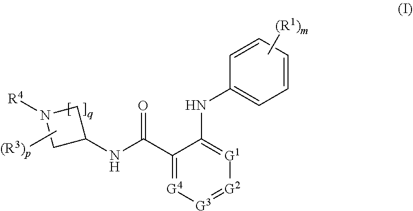

or a pharmaceutically acceptable salt, solvate or isotopic derivative thereof, wherein:
G¹ is CH or CR²
G², G³, and G⁴ are independently of each other N, CH, or CR²;
   provided that at least two of G², G³, and G⁴ are independently of each other CH or CR²; and further provided that when G¹ is CH, then at least one of G², G³, and G⁴ is N or CR²;
m is 0, 1, 2, 3, 4, or 5;
p is 0, 1, 2, 3, or 4;
q is 1, 2, or 3;
each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl);
each R² is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ haloalkyl, optionally substituted C₁-C₆ alkoxy, -[(optionally substituted C₁-C₆ alkylene)-(optionally substituted C₁-C₆ alkoxy)], —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl);

each R³ is independently selected from the group consisting of halogen, OH, NH₂, optionally substituted C₁-C₆ alkyl, optionally substituted C₁-C₆ haloalkyl, -[(optionally substituted alkylene)-(optionally substituted C₁-C₆ alkoxy)], optionally substituted C₁-C₆ alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl); and

R⁴ is selected from the group consisting of halogen, OH, NH₂, optionally substituted C₁-C₆ alkyl, optionally substituted C₁-C₆ haloalkyl, -[(optionally substituted C₁-C₆ alkylene)-(optionally substituted C₁-C₆ alkoxy)], optionally substituted C₁-C₆ alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl).

Provided in other aspects are compounds of Formula (I-1),

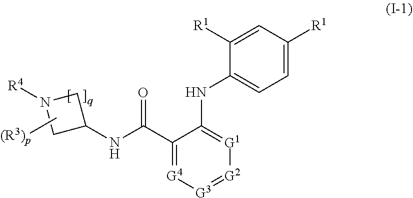

or a pharmaceutically acceptable salt, solvate or isotopic derivative thereof, wherein G¹, G², G³, G⁴, p, q, R¹, R², R³, and R⁴ are as defined for Formula (I).

Provided in other aspects are compounds of Formula (I-2a), (I-2b), or (I-2c):

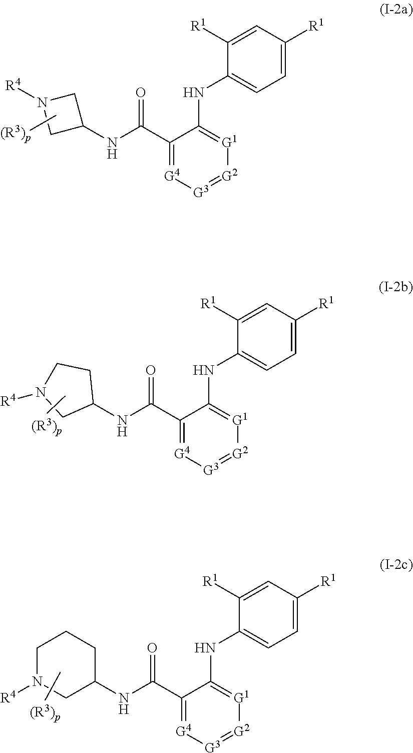

or a pharmaceutically acceptable salt, solvate or isotopic derivative thereof, wherein G¹, G², G³, G⁴, p, R¹, R², R³, and R⁴ are as defined for Formula (I).

Provided in other aspects are compounds of Formula (I-3a), (I-3b), or (I-3c):

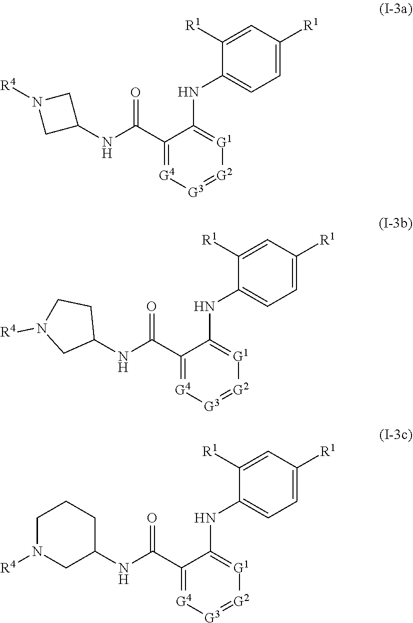

or a pharmaceutically acceptable salt, solvate or isotopic derivative thereof, wherein G¹, G², G³, G⁴, R¹, R², R³, and R⁴ are as defined for Formula (I).

Provided in some embodiments are compounds of Table 1, or a pharmaceutically acceptable salt, solvate or isotopic derivative thereof.

In some aspects, provided are pharmaceutical compositions containing a compound of any of the formulae described herein, or a pharmaceutically acceptable salt, solvate or isotopic derivative thereof, and a pharmaceutically acceptable diluent or carrier.

In some aspects, provided are combinations containing at least one compound of Formula (I), (I-1), (I-2a), (I-2b), (I-2c), (I-3a), (I-3b), (I-3c), or a compound of Table 1, or a pharmaceutically acceptable salt, solvate or isotopic derivative thereof, and a second prophylactic or therapeutic agent.

In some aspects, provided are compounds of Formula (I), such as compounds of Formula (I), (I-1), (I-2a), (I-2b), (I-2c), (I-3a), (I-3b), (I-3c), or a compound of Table 1, or a pharmaceutically acceptable salt, solvate or isotopic derivative thereof, for use in treating and/or preventing a proliferation disorder, such as a cancer, or a tumor in a subject. In some embodiments, the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

Provided in some aspects are methods of treating and/or preventing a proliferation disorder, such as a cancer, or a tumor in a subject, wherein the method includes administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein. In some embodiments, the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

In some aspects, the present disclosure provides use of at least one compound of any of the formulae described herein, or a pharmaceutically acceptable salt, solvate or isotopic derivative thereof, for the manufacture of a medicament.

In some aspects, the present disclosure provides a method for producing an anti-proliferative or anti-metastatic effect in a subject having a proliferation disorder, a cancer, or a tumor which is sensitive to inhibition of relevant kinases, such as MEK, including administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein.

In some aspects, provided are compounds of Formula (I), such as compounds of Formula (I), (I-1), (I-2a), (I-2b), (I-2c), (I-3a), (I-3b), (I-3c), or a compound of Table 1, or a pharmaceutically acceptable salt, solvate or isotopic derivative thereof, for use in the treatment of a neurodegenerative disease. In some embodiments, the neurodegenerative disease is selected from the group consisting of Amyotrophic lateral sclerosis, Parkinson's disease, Alzheimer's disease, and Huntington's disease.

In some aspects, the present disclosure provides a method for treating a neurodegenerative disease in a subject. In some embodiments, the method includes administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein. In some embodiments, the neurodegenerative disease is selected from the group consist of Amyotrophic lateral sclerosis, Parkinson's disease, Alzheimer's disease, and Huntington's disease.

In some aspects, the present disclosure provides a method for treating an immunodeficient disease in a subject. In some embodiments, the method includes administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein. In some embodiments, the immunodeficient disease is selected from the group consist of cancers, infectious disease, and some genetic diseases.

In yet another aspect, provided are methods for inhibiting an activity of one or more kinases, such as MEK, COT1, FGFR4, MINK, MYO3A, PKG1B, and PLK3, in a cell, including contacting the cell with an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein, wherein the contacting is in vitro, ex vivo, or in vivo.

### DETAILED DESCRIPTION

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

As used herein, reference to “about” a value or parameter herein includes (and describes) embodiments that are directed to that value or parameter per se. For example, description referring to “about X” includes description of “X”.

Unless clearly indicated otherwise, “an individual” or “a subject” as used herein intends a mammal, including but not limited to a human, bovine, primate, equine, canine, feline, porcine, and ovine animals. Thus, the compositions and methods provided herein have use in both human medicine and in the veterinary context, including use in agricultural animals and domestic pets. The individual may be a human who has been diagnosed with or is suspected of having a condition described herein, such as cancer. The individual may be a human who exhibits one or more symptoms associated with a condition described herein, such as cancer. The individual may be a human who has a mutated or abnormal gene associated with a condition described herein, such as cancer. The individual may be a human who is genetically or otherwise predisposed to or at risk of developing a condition described herein, such as cancer.

As used herein, “treatment” or “treating” is an approach for obtaining beneficial or desired results including clinical results. For purposes of the compositions and methods provided herein, beneficial or desired clinical results include, but are not limited to, one or more of the following: decreasing one or more symptoms resulting from the condition, diminishing the extent of the condition, stabilizing the condition (e.g., preventing or delaying the worsening of the condition), preventing or delaying the spread (e.g., metastasis) of the condition, delaying or slowing the progression of the condition, ameliorating a disease state, providing a remission (whether partial or total) of a disease, decreasing the dose of one or more other medications required to treat the condition, enhancing the effect of another medication used to treat the condition, increasing the quality of life of an individual having the condition, and/or prolonging survival. A method of treating cancer encompasses a reduction of the pathological consequence of cancer. The methods described herein contemplate any one or more of these aspects of treatment.

As used herein, an “at risk” individual is an individual who is at risk of developing a disease or condition described herein, such as cancer. An individual “at risk” may or may not have detectable disease, and may or may not have displayed detectable disease prior to the treatment methods described herein. “At risk” denotes that an individual has one or more so-called risk factors, which are measurable parameters that correlate with development of a disease or condition described herein, such as cancer. An individual having one or more of these risk factors has a higher probability of developing the disease or condition than an individual without these risk factor(s).

As used herein, by “combination therapy” is meant a therapy that includes two or more different compounds. Thus, in one aspect, a combination therapy comprising a compound detailed herein and another compound is provided. In some variations, the combination therapy optionally includes one or more pharmaceutically acceptable carriers or excipients, non-pharmaceutically active compounds, and/or inert substances. In various embodiments, treatment with a combination therapy may result in an additive or even synergistic (e.g., greater than additive) result compared to administration of a single compound provided herein alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency, and/or duration of one or more side-effects associated with the compound.

As used herein, the term “effective amount” intends such amount of a compound provided herein which in combination with its parameters of efficacy and toxicity, should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds. In various embodiments, an effective amount of the composition or therapy may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of a disease or condition described herein, such as cancer.

As is understood in the art, an “effective amount” may be in one or more doses, i.e., a single dose or multiple doses may be required to achieve the desired treatment endpoint. An effective amount may be considered in the context of administering one or more therapeutic agents, and a compound, or pharmaceutically acceptable salt thereof, may be considered to be given in an effective amount if, in conjunction with one or more other agents, a desirable or beneficial result may be or is achieved.

A “therapeutically effective amount” refers to an amount of a compound or salt thereof sufficient to produce a desired therapeutic outcome (e.g., reducing the severity or duration of, stabilizing the severity of, or eliminating one or more symptoms of a disease or condition described herein, such as cancer). For therapeutic use, beneficial or desired results include, e.g., decreasing one or more symptoms resulting from the disease (biochemical, histologic and/or behavioral), including its complications and intermediate pathological phenotypes presenting during development of the disease or condition, increasing the quality of life of those suffering from the disease or condition, decreasing the dose of other medications required to treat the disease or condition, enhancing effect of another medication, delaying the progression of the disease or condition, and/or prolonging survival of patients.

It is understood that an effective amount of a compound or pharmaceutically acceptable salt thereof, including a prophylactically effective amount, may be given to an individual in the adjuvant setting, which refers to a clinical setting in which an individual has had a history of cancer, and generally (but not necessarily) has been responsive to therapy, which includes, but is not limited to, surgery (e.g., surgical resection), radiotherapy, and chemotherapy. However, because of their history of cancer, these individuals are considered at risk of developing cancer. Treatment or administration in the “adjuvant setting” refers to a subsequent mode of treatment.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound provided herein in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound provided herein as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Alkyl” refers to and includes saturated linear or branched univalent hydrocarbon structures and combinations thereof. Particular alkyl groups are those having 1 to 20 carbon atoms (a “C₁-C₂₀ alkyl”). More particular alkyl groups are those having 1 to 8 carbon atoms (a “C₁-C₈ alkyl”) or 1 to 6 carbon atoms (a “C₁-C₆ alkyl”). When an alkyl residue having a specific number of carbons is named, all geometric isomers having that number of carbons are intended to be encompassed and described; thus, for example, “butyl” is meant to include n-butyl, sec-butyl, iso-butyl, and tent-butyl; “propyl” includes n-propyl and iso-propyl. This term is exemplified by groups such as methyl, t-butyl, n-heptyl, octyl, and the like.

“Alkenyl” refers to an unsaturated hydrocarbon group having at least one site of olefinic unsaturation (i.e., having at least one moiety of the formula C═C) and preferably having from 2 to 10 carbon atoms and more preferably 2 to 8 carbon atoms. Examples of alkenyl include but are not limited to —CH₂—CH═CH—CH₃ and —CH═CH—CH═CH₂.

“Alkynyl” refers to an unsaturated hydrocarbon group having at least one site of acetylenic unsaturation (i.e., having at least one moiety of the formula C═C) and preferably having from 2 to 10 carbon atoms and more preferably 2 to 8 carbon atoms and the like.

The term “alkoxy” refers to an —O-alkyl group, where the O is the point of attachment to the rest of the molecule, and alkyl is as defined above.

The term “thioalkoxy” refers to an —S-alkyl group, where the S is the point of attachment to the rest of the molecule, and alkyl is as defined above.

“Haloalkyl” refers to an alkyl group with one or more halo sub stituents, such as one, two, three, four, five, six, seven, eight, or nine halo substituents. Examples of haloalkyl groups include —CF₃, —(CH₂)F, —CHF₂, CH₂Br, —CH₂CF₃, —CH₂CHF₂, and —CH₂CH₂F.

“Carbocycle”, “carbocyclic”, or “carbocyclyl” refers to a saturated or an unsaturated non-aromatic cyclic hydrocarbon group having a single ring or multiple condensed rings having from 3 to 13 annular carbon atoms. A carbocycle comprising more than one ring may be fused, spiro or bridged, or any combination thereof. In fused ring systems, one or more of the rings can be aryl. A carbocycle having more than one ring where at least one ring is aromatic may be connected to the parent structure at either a non-aromatic ring position or at an aromatic ring position. In one variation, a carbocycle having more than one ring where at least one ring is aromatic is connected to the parent structure at a non-aromatic ring position.

“Heterocycle”, “heterocyclic”, or “heterocyclyl” refers to a saturated or an unsaturated non-aromatic group having a single ring or multiple condensed rings, and having from 1 to 10 annular carbon atoms and from 1 to 4 annular heteroatoms, such as nitrogen, sulfur or oxygen, and the like. A heterocycle comprising more than one ring may be fused, spiro or bridged, or any combination thereof. In fused ring systems, one or more of the rings can be aryl or heteroaryl. A heterocycle having more than one ring where at least one ring is aromatic may be connected to the parent structure at either a non-aromatic ring position or at an aromatic ring position. In one variation, a heterocycle having more than one ring where at least one ring is aromatic is connected to the parent structure at a non-aromatic ring position.

“Aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” or “HetAr” refers to an unsaturated aromatic carbocyclic group having from 1 to 10 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

The term “halogen” represents chlorine, fluorine, bromine, or iodine. The term “halo” represents chloro, fluoro, bromo, or iodo. The terms “halogen” and “halo” are understood to be equivalent and may be used interchangeably when referring to a substituent group.

The term “substituted” means that the specified group or moiety bears one or more substituents including, but not limited to, substituents such as alkoxy, acyl, acyloxy, carbonylalkoxy, acylamino, amino, aminoacyl, aminocarbonyl amino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, carbocyclyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

A composition of “substantially pure” compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

Any formula given herein is intended to represent compounds having structures depicted by the structural formula as well as certain variations or forms. In particular, compounds of any formula given herein, such compound of Formula (I), (I-1), (I-2a), (I-2b), (I-2c), (I-3a), (I-3b), or (I-3c), may have asymmetric centers and therefore exist in different enantiomeric forms. These steromeric mixtures can be separated into their individual stereomers on the basis of their physical chemical or optical differences by methods known to those skilled in the art, for example, by chromatography or fractional crystallization. All such isomers, including diastereomers and enantiomers are considered as part of the invention. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, chlorine, and iodine, such as ²H, ³H, ¹¹C, ¹³C, ¹⁴C, ¹⁵N , ¹⁸O, ¹⁷O, ³¹P, ³²P, ³⁵S, ¹⁸F, ³⁶Cl, and ¹²⁵I, respectively. Substitution with heavier isotopes such as deuterium (i.e., ²H) may afford certain therapeutic advantages resulting from greater metabolic stability, for example increased in vivo half-life or reduced dosage requirements. Isotopically labeled compounds described herein and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

### Exemplary Compounds

Compounds and salts thereof (such as pharmaceutically acceptable salts) are detailed herein, including in the Summary and in the appended claims. Also provided are the use of all of the compounds described herein, including any and all stereoisomers, including geometric isomers (cis/trans), E/Z isomers, enantiomers, diastereomers, and mixtures thereof in any ratio including racemic mixtures, salts and solvates of the compounds described herein, as well as methods of making such compounds. Any compound described herein may also be referred to as a drug.

In one aspect, provided are compounds of Formula (I):

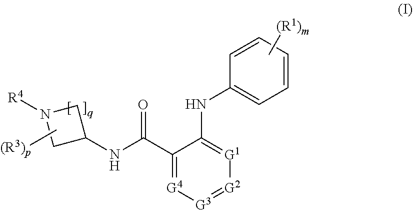

or a pharmaceutically acceptable salt, solvate or isotopic derivative thereof, wherein:
G¹ is CH or CR²
G², G³, and G⁴ are independently of each other N, CH, or CR²; provided that at least two of G², G³, and G⁴ are independently of each other CH or CR²; and further provided that when G¹ is CH, then at least one of G², G³, and G⁴ is N or CR²;
m is 0, 1, 2, 3, 4, or 5;
p is 0, 1, 2, 3, or 4;
q is 1, 2, or 3;
each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl);

each R² is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ haloalkyl, optionally substituted C₁-C₆ alkoxy, -[(optionally substituted C₁-C₆ alkylene)-(optionally substituted C₁-C₆ alkoxy)], —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)2, —NH[(optionally substituted C₁-C₆ alkylene)-(optionally substituted C₁-C₆ alkoxy)], —N[(optionally substituted C₁-C₆ alkylene)-(optionally substituted C alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl);

each R³ is independently selected from the group consisting of halogen, OH, NH₂, optionally substituted C₁-C₆ alkyl, optionally substituted C₁-C₆ haloalkyl, -[(optionally substituted C₁-C₆ alkylene)-(optionally substituted C₁-C₆ alkoxy)], optionally substituted C alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)- (optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl); and

R⁴ is selected from the group consisting of halogen, OH, NH₂, optionally substituted C₁-C₆ alkyl, optionally substituted C₁-C₆ haloalkyl, -[(optionally substituted C₁-C₆ alkylene)-(optionally substituted C₁-C₆ alkoxy)], optionally substituted C₁-C₆ alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl).

In some embodiments of the compounds of Formula (I), m is 0. In some embodiments of the compounds of Formula (I), m is 1. In some embodiments of the compounds of Formula (I), m is 2. In some embodiments of the compounds of Formula (I), m is 3. In some the compounds of Formula (I), m is 4. In some embodiments of the compounds of Formula (I), m is 5.

In some embodiments, the compound of Formula (I) is a compound of Formula (I-1):

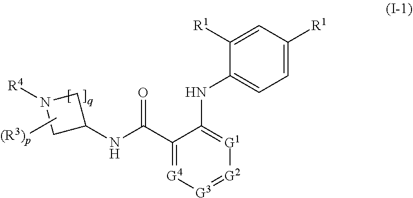

or a pharmaceutically acceptable salt, solvate or isotopic derivative thereof, wherein G¹, G², G³, G⁴, p, q, R¹, R², R³, and R⁴ are as defined for Formula (I).

In some embodiments of the compounds of Formula (I) and (I-1), q is 1. In some the compounds of Formula (I) or (I-1), q is 2. In some embodiments of the compounds of Formula (I) or (I-1), q is 3.

In some embodiments, the compound of Formula (I) is a compound of Formula (I-2a), (I-2b), or (I-2c):

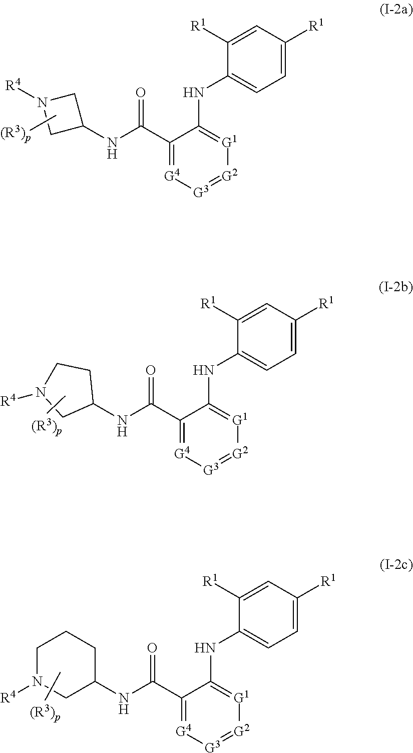

or a pharmaceutically acceptable salt, solvate or isotopic derivative thereof, wherein G¹, G², G³, G⁴, p, R¹, R², R³, and R⁴ are as defined for Formula (I).

In some embodiments of the compounds of Formula (I), (I-1), (I-2a), (I-2b), and (I-2c), p is 0. In some embodiments of the compounds of Formula (I), (I-1), (I-2a), (I-2b), and (I-2c), p is 1. In some embodiments of the compounds of Formula (I), (I-1), (I-2a), (I-2b), and (I-2c), p is 2. In some embodiments of the compounds of Formula (I), (I-1), (I-2a), (I-2b), and (I-2c), p is 3. In some embodiments of the compounds of Formula (I), (I-1), (I-2a), (I-2b), and (I-2c), p is 4.

In some embodiments, the compound of Formula (I) is a compound of Formula (I-3a), (I-3b), or (I-3c):

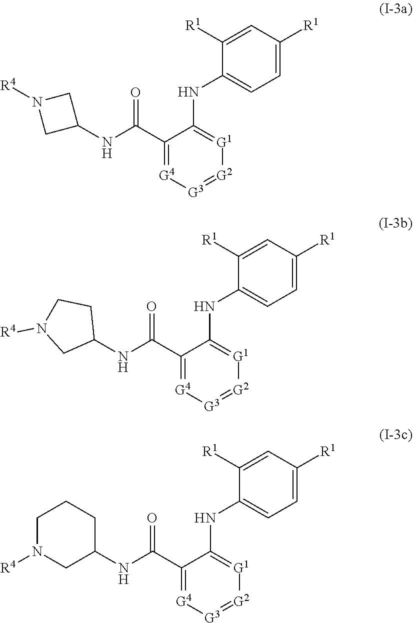

or a pharmaceutically acceptable salt, solvate or isotopic derivative thereof, wherein G¹, G², G³, G⁴, R¹, R², R³, and R⁴ are as defined for Formula (I).

In some embodiments of the compounds of Formula (I), (I-1), (I-2a), (I-2b), (I-2c), (I-3a), (I-3b), or (I-3c), G¹ is CH or CR². In some embodiments, G¹ is CH. In some embodiments, G¹ is CR². In some embodiments, G¹ is CR² and the R² of G¹ is halogen. In some embodiments, G¹ is CR² and the R² of G¹ is fluoro.

In some embodiments of the compounds of Formula (I), (I-1), (I-2a), (I-2b), (I-2c), (I-3a), (I-3b), or (I-3c), G² is N, CH, or CR². In some embodiments, G² is N. In some embodiments, G² is CH or CR². In some embodiments, G² is CH. In some embodiments, G² is CR². In some embodiments, G² is CR² and the R² of G² is halogen. In some embodiments, G² is CR² and the R² of G² is fluoro.

In some embodiments of the compounds of Formula (I), (I-1), (I-2a), (I-2b), (I-2c), (I-3a), (I-3b), or (I-3c), G³ is N, CH, or CR². In some embodiments, G³ is N. In some embodiments, G³ is CH or CR². In some embodiments, G³ is CH. In some embodiments, G³ is CR². In some embodiments, G³ is CR² and the R² of G³ is halogen. In some embodiments, G³ is CR² and the R² of G³ is fluoro.

In some embodiments of the compounds of Formula (I), (I-1), (I-2a), (I-2b), (I-2c), (I-3a), (I-3b), or (I-3c), G⁴ is N, CH, or CR². In some embodiments, G⁴ is N. In some embodiments, G⁴ is CH or CR². In some embodiments, G⁴ is CH. In some embodiments, G⁴ is CR². In some embodiments, G⁴ is CR² and the R² of G⁴ is halogen. In some embodiments, G⁴ is CR⁴ and the R² of G² is fluoro. In some embodiments, G⁴ is CR⁴ and the R² of G² is chloro.

In some embodiments of the compounds of Formula (I), (I-1), (I-2a), (I-2b), (I-2c), (I-3a), (I-3b), or (I-3c), G¹is CH, G² is CF, G³ is CH, and G⁴ is CF.

In some embodiments of the compounds of Formula (I), (I-1), (I-2a), (I-2b), (I-2c), (I-3a), (I-3b), or (I-3c), G¹ is CF, G² is CF, G³ is CF, and G⁴ is CF.

In some embodiments of the compounds of Formula (I), (I-1), (I-2a), (I-2b), (I-2c), (I-3a), (I-3b), or (I-3c), G¹ is CH, G² is N, G³ is CH, and G⁴ is CH.

In some embodiments of the compounds of Formula (I), (I-1), (I-2a), (I-2b), (I-2c), (I-3a), (I-3b), or (I-3c), G¹- is CH, G² is N, G³ is CH, and G⁴ is CF.

In some embodiments of the compounds of Formula (I), (I-1), (I-2a), (I-2b), (I-2c), (I-3a), (I-3b), or (I-3c), CH, G² is N, G³ is CH, and G⁴ is CC_{1.}

In some embodiments of the compounds of Formula (I), (I-1), (I-2a), (I-2b), (I-2c), (I-3a), (I-3b), or (I-3c), CH, G² is CH, G³ is N, and G⁴ is CH.

In some embodiments of the compounds of Formula (I), (I-1), (I-2a), (I-2b), (I-2c), (I-3a), (I-3b), or (I-3c), m is 0.

In some embodiments of the compounds of Formula (I), (I-1), (I-2a), (I-2b), (I-2c), (I-3a), (I-3b), or (I-3c), m is 1. In some embodiments, R¹ is selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl). In some embodiments, R¹ is selected from the group consisting of halogen, optionally substituted C₂-C₆ alkynyl, and —C(═O)-(optionally substituted C₁-C₆ alkyl). In some embodiments, R¹ is halogen. In some embodiments, R¹ is fluoro. In some embodiments, R¹ is chloro. In some embodiments, R¹ is bromo. In some embodiments, R¹ is iodo. In some embodiments, R¹ is optionally substituted C₂-C₆ alkynyl. In some embodiments, R¹ is ethynyl. In some embodiments, R¹ is —C(═O)-(optionally substituted C₁-C₆ alkyl). In some embodiments, R¹ is —C(═O)-CH_{3.}

In some embodiments of the compounds of Formula (I), (I-1), (I-2a), (I-2b), (I-2c), (I-3a), (I-3b), or (I-3c), m is 2. In some embodiments, each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl). In some embodiments, each R¹ is independently selected from the group consisting of halogen, optionally substituted C₂-C₆ alkynyl, and —C(═O)-(optionally substituted C₁-C₆ alkyl). In some embodiments, each R¹ is indepdently selected from the group consisting of fluoro, chloro, bromo, and iodo. In some embodiments, one R¹ is fluoro and the other R¹ is iodo. In some embodiments, one R¹ is chloro and the other R¹ is bromo. In some embodiments, one R¹ is halogen and the other R¹ is optionally substituted C₂-C₆ alkynyl. In some embodiments, one R¹ is fluoro and the other R¹ is ethynyl. In some embodiments, one R¹ is halogen and the other R¹ is —C(═O)-(optionally substituted C₁-C₆ alkyl). In some embodiments, one R¹ is fluoro and the other R¹ is —C(═O)—CH₃.

In some embodiments of the compounds of Formula (I), (I-1), (I-2a), (I-2b), (I-2c), (I-3a), (I-3b), or (I-3c), m is 3. In some embodiments, each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl). In some embodiments, each R¹ is independently selected from the group consisting of halogen, optionally substituted C₂-C₆ alkynyl, and —C(═O)-(optionally substituted C₁-C₆ alkyl). In some embodiments, each R¹ is indepdently selected from the group consisting of fluoro, chloro, bromo, and iodo.

In some embodiments of the compounds of Formula (I), (I-1), (I-2a), (I-2b), (I-2c), (I-3a), (I-3b), or (I-3c), m is 4. In some embodiments, each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl). In some embodiments, each R¹ is independently selected from the group consisting of halogen, optionally substituted C₂-C₆ alkynyl, and —C(═O)-(optionally substituted C₁-C₆ alkyl). In some embodiments, each R¹ is indepdently selected from the group consisting of fluoro, chloro, bromo, and iodo.

In some embodiments of the compounds of Formula (I), (I-1), (I-2a), (I-2b), (I-2c), (I-3a), (I-3b), or (I-3c), m is 5. In some embodiments, each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)] , —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)] , —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl). In some embodiments, each R¹ is independently selected from the group consisting of halogen, optionally substituted C₂-C₆ alkynyl, and —C(═O)-(optionally substituted C₁-C₆ alkyl). In some embodiments, each R¹ is indepdently selected from the group consisting of fluoro, chloro, bromo, and iodo.

In some embodiments of the compounds of Formula (I), (I-1), (I-2a), (I-2b), (I-2c), (I-3a), (I-3b), or (I-3c), R⁴ is selected from the group consisting of hydrogen, halogen, OH, NH₂, optionally substituted C₁-C₆ alkyl, optionally substituted C₁-C₆ haloalkyl, -[(optionally substituted C₁-C₆ alkylene)-(optionally substituted C₁-C₆ alkoxy)], optionally substituted C₁-C₆ alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl). In some embodiments, R⁴ is selected from the group consisting of hydrogen, optionally substituted C₁-C₆ alkyl, optionally substituted C₁-C₆ haloalkyl, and -[(optionally substituted C₁-C₆ alkylene)-(optionally substituted C₁-C₆ alkoxy)]. In some embodiments, R⁴ is selected from the group consisting of hydrogen, optionally substituted C₁-C₆ alkyl, optionally substituted C₁-C₆ haloalkyl, -[(optionally substituted C₁-C₆ alkylene)-(optionally substituted C₁-C₆ alkoxy)], and optionally substituted C₁-C₆ alkylene-COOH. In some embodiments, R⁴ is selected from the group consisting of methyl, ethyl, prop-2-yl, 2-fluoroeth-1-yl, 2-methoxyeth-1-yl, —CH₂—CH₂—COOH and —CH₂—CH₂—CH₂—COOH. In some embodiments, R⁴ is hydrogen. In some embodiments, R⁴ is optionally substituted C₁-C₆ alkyl. In some embodiments, R⁴ is selected from the group consisting of methyl, ethyl, and prop-2-yl. In some embodiments, R⁴ is methyl. In some embodiments, R⁴ is ethyl. In some embodiments, R⁴ is ethyl prop-2-yl. In some embodiments, R⁴ is optionally substituted C₁-C₆ haloalkyl. In some embodiments, R⁴ is 2-fluoroeth-1-yl. In some embodiments, R⁴ is -[(optionally substituted C₁-C₆ alkylene)-(optionally substituted C₁-C₆ alkoxy)]. In some embodiments, R⁴ is 2-methoxyeth-1-yl. In some embodiments, R⁴ is optionally substituted C₁-C₆ alkylene-COOH. In some embodiments, R⁴ is —CH₂—CH₂—COOH. In some embodiments, R⁴ is —CH₂—CH₂—CH₂—COOH.

In some embodiments, provided herein are compounds of Formula (I), (I-1), (I-2a), (I-2b), (I-2c), (I-3a), (I-3b), and (I-3c), or pharmaceutically acceptable salts thereof.

In some embodiments, provided herein are compounds and salts thereof described in Table 1, and uses thereof.

TABLE 1

| Compound | | |
|---|---|---|
| No. | Structure | Chemical Name |
| 1 | 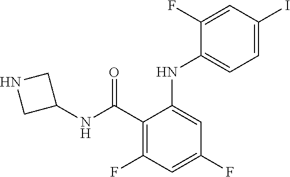 [SMILES: O=C(NC1CNC1)c1c(F)cc(F)cc1Nc1ccc(I)cc1F] | N-(azetidin-3-yl)-2,4- difluoro-6-((2-fluoro-4- iodophenyl)amino)benzamide |
| 2 | 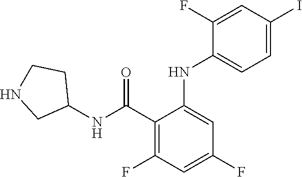 [SMILES: O=C(NC1CCNC1)c1c(F)cc(F)cc1Nc1ccc(I)cc1F] | 2,4-difluoro-6-((2-fluoro-4- iodophenyl)amino)-N- (pyrrolidin-3-yl)benzamide |
| 3 | 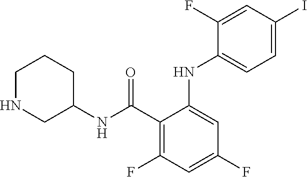 [SMILES: O=C(NC1CCCNC1)c1c(F)cc(F)cc1Nc1ccc(I)cc1F] | 2,4-difluoro-6-((2-fluoro-4- iodophenyl)amino)-N- (piperidin-3-yl)benzamide |
| 4 | 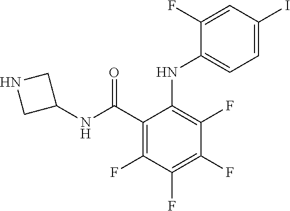 [SMILES: O=C(NC1CNC1)c1c(F)c(F)c(F)c(F)c1Nc1ccc(I)cc1F] | N-(azetidin-3-yl)-2,3,4,5- tetrafluoro-6-((2-fluoro-4- iodophenyl)amino)benzamide |
| 5 | 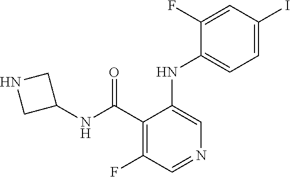 [SMILES: O=C(NC1CNC1)c1c(F)cncc1Nc1ccc(I)cc1F] | N-(azetidin-3-y1)-3-fluoro-5- ((2-fluoro-4- iodophenyl)amino) isonicotinamide |
| 6 | 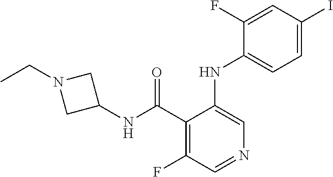 [SMILES: CCN1CC(NC(=O)c2c(F)cncc2Nc2ccc(I)cc2F)C1] | N-(1-ethylazetidin-3-y1)-3- fluoro-5-((2-fluoro-4- iodophenyl)amino) isonicotinamide |
| 7 | 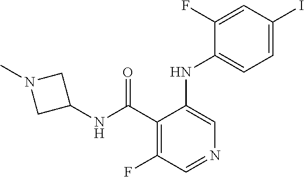 [SMILES: CN1CC(NC(=O)c2c(F)cncc2Nc2ccc(I)cc2F)C1] | 3-fluoro-5-((2-fluoro-4- iodophenyl)amino)-N-(1- methylazetidin-3- yl)isonicotinamide |
| 8 | 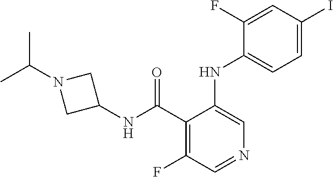 [SMILES: CC(C)N1CC(NC(=O)c2c(F)cncc2Nc2ccc(I)cc2F)C1] | 3-fluoro-5-((2-fluoro-4- iodophenyl)amino)-N-(1- isopropylazetidin-3- yl)isonicotinamide |
| 9 | 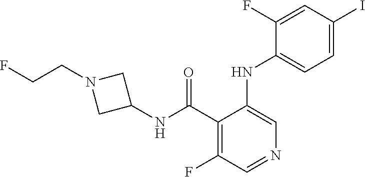 [SMILES: O=C(NC1CN(CCF)C1)c1c(F)cncc1Nc1ccc(I)cc1F] | 3-fluoro-5-((2-fluoro-4- iodophenyl)amino)-N-(1-(2- fluoroethyl)azetidin-3- yl)isonicotinamide |
| 10 | 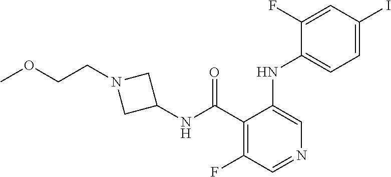 [SMILES: COCCN1CC(NC(=O)c2c(F)cncc2Nc2ccc(I)cc2F)C1] | 3-fluoro-5-((2-fluoro-4- iodophenyl)amino)-N-(1-(2- methoxyethyl)azetidin-3- yl)isonicotinamide |
| 11 | 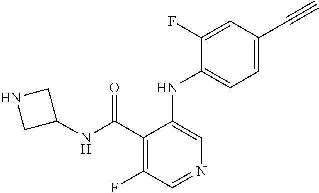 [SMILES: C#Cc1ccc(Nc2cncc(F)c2C(=O)NC2CNC2)c(F)c1] | N-(azetidin-3-y1)-3-((4- ethyny1-2- fluorophenyl)amino)-5- fluoroisonicotinamide |
| 12 | 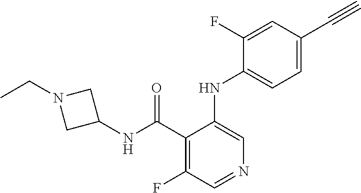 [SMILES: C#Cc1ccc(Nc2cncc(F)c2C(=O)NC2CN(CC)C2)c(F)c1] | N-(1-ethylazetidin-3-y1)-3- ((4-ethyny1-2- fluorophenyl)amino)-5- fluoroisonicotinamide |
| 13 | 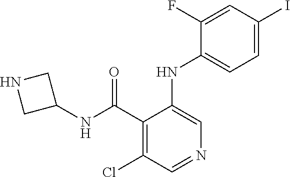 [SMILES: O=C(NC1CNC1)c1c(Cl)cncc1Nc1ccc(I)cc1F] | N-(azetidin-3-y1)-3-chloro-5- ((2-fluoro-4- iodophenyl)amino) isonicotinamide |
| 14 | 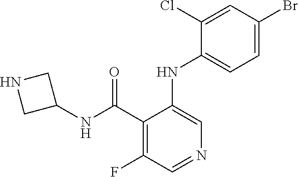 [SMILES: O=C(NC1CNC1)c1c(F)cncc1Nc1ccc(Br)cc1Cl] | N-(azetidin-3-y1)-3-((4- bromo-2- chlorophenyl)amino)-5- fluoroisonicotinamide |
| 15 | 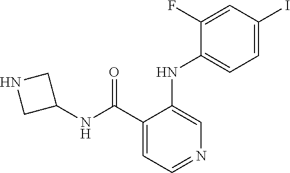 [SMILES: O=C(NC1CNC1)c1ccncc1Nc1ccc(I)cc1F] | N-(azetidin-3-y1)-3-((2- fluoro-4- iodophenyl)amino) isonicotinamide |
| 16 | 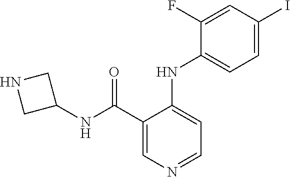 [SMILES: O=C(NC1CNC1)c1cnccc1Nc1ccc(I)cc1F] | N-(azetidin-3-y1)-4-((2- fluoro-4- iodophenyl)amino) nicotinamide |
| 17 | 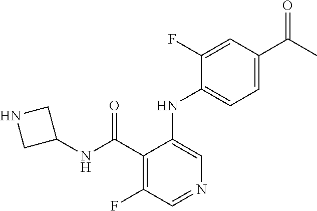 [SMILES: CC(=O)c1ccc(Nc2cncc(F)c2C(=O)NC2CNC2)c(F)c1] | 3-((4-acetyl-2- fluorophenyl)amino)-N- (azetidin-3-y1)-5- fluoroisonicotinamide |
| 18 | 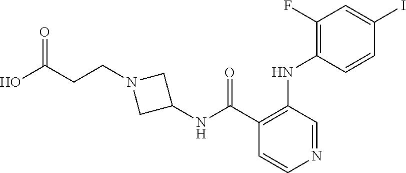 [SMILES: O=C(O)CCN1CC(NC(=O)c2ccncc2Nc2ccc(I)cc2F)C1] | 3-(3-(3-((2-fluoro-4- iodophenyl)amino) isonicotinamido) azetidin-1-yl)propanoic acid |
| 19 | 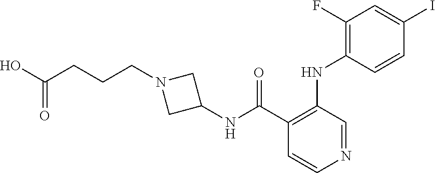 [SMILES: O=C(O)CCCN1CC(NC(=O)c2ccncc2Nc2ccc(I)cc2F)C1] | 4-(3-(3-((2-fluoro-4- iodophenyl)amino) isonicotinamido)azetidin- 1-yl)butanoic acid |
| 20 | 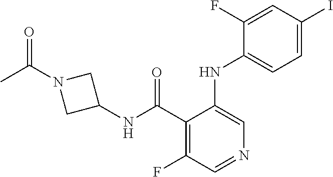 [SMILES: CC(=O)N1CC(NC(=O)c2c(F)cncc2Nc2ccc(I)cc2F)C1] | N-(1-acetylazetidin-3-yl)-3- fluoro-5-((2-fluoro-4- iodophenyl)amino) isonicotinamide |

and pharmaceutically acceptable salts thereof.

Any formula or compound given herein, such as Formula (I), (I-1), (I-2a), (I-2b), (I-2c), (I-3a), (I-3b), or (I-3c), or compounds of Table 1, is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 has two or more stereocenters, also provided are any enantiomer or diastereomer of the compound. Furthermore, certain structures may exist as geometric isomers (i.e., cis and trans isomers), as tautomers, or as atropisomers. Additionally, any compound of Table 1 is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein, such as Formula (I), (I-1), (I-2a), (I-2b), (I-2c), (I-3a), (I-3b), or (I-3c), is intended to refer to hydrates, solvates, and amorphous forms of such compounds, and mixtures thereof, even if such forms are not listed explicitly. In some embodiments, the solvent is water and the solvates are hydrates.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of G¹, G², G³, G⁴, m, p, q, R¹, R², R³, and R⁴ provided herein can be combined with every other variation or embodiment of G¹, G², G³, G⁴, m, p, q, R¹, R², R³, and R⁴, as if each combination had been individually and specifically described.

### Compositions

Also provided are compositions, such as pharmaceutical compositions, that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, the present disclosure provides for a pharmaceutical composition comprising a compound described above admixed with at least one pharmaceutically acceptable carrier or excipient. In some embodiments, provided are compositions, such as pharmaceutical compositions that contain one or more compounds described herein, or a pharmaceutically acceptable salt thereof

In some embodiments, provided is a pharmaceutically acceptable composition comprising a compound of Formula (I), (I-1), (I-2a), (I-2b), (I-2c), (I-3a), (I-3b), (I-3c), or a compound of Table 1, or a pharmaceutically acceptable salt thereof In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds that are substantially pure.

Also provided are packaged pharmaceutical compositions, comprising a pharmaceutical composition as described herein and instructions for using the composition to treat a patient suffering from a disease or condition described herein.

### Pharmaceutical Formulations

The present disclosure also provides a composition, e.g., a pharmaceutical composition, containing one or more of the compounds described herein, formulated together with a pharmaceutically acceptable carrier. Pharmaceutical compositions of the invention also can be administered in combination therapy, i.e., combined with other agents. For example, the combination therapy can include a compound as described herein combined with at least one other active agent.

Pharmaceutically acceptable carriers may include any and all carriers, excipients, stabilizers, solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the compound described herein, may be coated in a material to protect the compound from the action of acids and other natural conditions that may inactivate the compound.

Acceptable carriers, excipients, or stabilizers are nontoxic to recipients at standard dosages and concentrations to be administered, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ or polyethylene glycol (PEG).

The pharmaceutical compositions of the invention may include one or more pharmaceutically acceptable salts. A pharmaceutically acceptable salt retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The compounds having formula (I), (I-1), (I-2a), (I-2b), (I-2c), (I-3a), (I-3b), and (I-3c) as described herein are generally soluble in organic solvents such as chloroform, dichloromethane, ethyl acetate, ethanol, methanol, isopropanol, acetonitrile, glycerol, N,N-dimethylformamide, N,N-dimethylacetamide, dimethylsulfoxide, etc. In one embodiment, the present invention provides formulations prepared by mixing a compound having formula (I), (I-1), (I-2a), (I-2b), (I-2c), (I-3a), (I-3b), and (I-3c) with a pharmaceutically acceptable carrier. In one aspect, the formulation may be prepared using a method comprising: a) dissolving a described compound in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

In some embodiments, a compound or salt thereof described herein or a composition described herein may be used in a method of treating musculoskeletal disease. In some embodiments, skeletal muscle mass, quality and/or strength are increased. In some embodiments, synthesis of muscle proteins is increased. In some embodiments, skeletal muscle fiber atrophy is inhibited.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug Combinations

The methods of the embodiments comprise administering an effective amount of at least one exemplary compound of the present disclosure; optionally the compound may be administered in combination with one or more additional therapeutic agents. In some embodiments, the additional therapeutic agent is known to be useful for treating a proliferation disorder, such as a cancer, or a tumor in a subject. In some embodiments, the additional therapeutic agent is known to be useful for treating a neurodegenerative disorder. In some embodiments, the additional therapeutic agent is known to be useful for treating an immunodeficient disease. In some embodiments, the additional therapeutic agent is an anticancer drug selected from the group consisting of RAS inhibitors, RAF inhibitors, and ERK inhibitors. In some embodiments, the additional therapeutic agent is an immunotherapy, such as PD-1 antibodies.

The additional active ingredients may be administered in a separate pharmaceutical composition from at least one exemplary compound of the present disclosure or may be included with at least one exemplary compound of the present disclosure in a single pharmaceutical composition. The additional active ingredients may be administered simultaneously with, prior to, or after administration of at least one exemplary compound of the present disclosure.

### Dosages and Dosage Forms

For the prevention or treatment of disease, the appropriate dosage of compounds described herein will depend on the type of disease to be treated, the severity and course of the disease, whether the compound is administered for preventive or therapeutic purposes, mode of delivery, previous therapy, and the subject's clinical history. The compounds described herein are suitably administered to a subject at one time or over a series of treatments. Depending on the type and severity of the disease, a typical daily dosage might range from about 0.0001 mg/kg to 100 mg/kg or more, depending on the factors mentioned above. For repeated administrations over several days or longer, depending on the condition, the treatment is sustained until a desired suppression of disease symptoms occurs.

For example dosages can be 0.3 mg/kg body weight, 1 mg/kg body weight, 3 mg/kg body weight, 5 mg/kg body weight or 10 mg/kg body weight or within the range of 1-10 mg/kg. Treatment regimens may comprise administration once per week, once every two weeks, once every three weeks, once every four weeks, once per month, once every 3 months or once every three to 6 months. In other embodiments, sustained release formulations are administered, which would result in less frequent administration compared to non-sustained release formulations.

The amount of active ingredient which can be combined with a carrier material to produce a single dosage form will generally be that amount of the composition which produces a therapeutic effect, without being toxic to the subject. Generally, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

### Administration

A composition described herein can be administered via one or more routes of administration using one or more of a variety of methods known in the art. As will be appreciated by the skilled artisan, the route and/or mode of administration will vary depending upon the desired results. Routes of administration for the compounds and compositions described herein include oral, sublingual, buccal, intranasal, topical, rectal, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

### Methods of Treatment

The compounds and pharmaceutical compositions herein may be used for any suitable purpose. For example, the present compounds can be used in therapy and/or testing.

The compounds and pharmaceutical compositions herein may be used to treat and/or prevent a proliferation disorder, such as a cancer, or a tumor in an individual. In some embodiments, provided are methods of treating or preventing a proliferation disorder, such as a cancer, or a tumor in an individual, comprising administering to the individual in need thereof a compound of Formula (I), (I-1), (I-2a), (I-2b), (I-2c), (I-3a), (I-3b), (I-3c), or a compound of Table 1, or a pharmaceutically acceptable salt thereof In some embodiments, provided are methods of treating or preventing a proliferation disorder, such as a cancer, or a tumor in a subject in need thereof comprising administering to the subject a therapeutically effective amount of at least one chemical entity as described herein.

In some embodiments, the compounds of Formula (I), (I-1), (I-2a), (I-2b), (I-2c), (I-3a), (I-3b), (I-3c), or compounds of Table 1, or a pharmaceutically acceptable salt thereof, are inhibitors of one or more kinases selected from the group consisting of MEK, COT1, FGFR4, MINK, MYO3A, PKG1B, and PLK3, and thus are all adapted to therapeutic use as antiproliferative or anti-metastatic agents (e.g., anticancer) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin (e.g., psoriasis) and benign hyperplasia of the prostate (e.g., BPH). In addition, it is expected that a compound of the present invention may possess activity against brain metastases originated from these disorders.

In some embodiments, compounds of Formula (I), (I-1), (I-2a), (I-2b), (I-2c), (I-3a), (I-3b), (I-3c), or compounds of Table 1, or a pharmaceutically acceptable salt thereof, may also be useful in the treatment of additional disorders in which aberrant expression ligand/receptor interactions or activation or signaling events related to various kinases, are involved. Such disorders may include those of neuronal, glial, astrocytal, hypothalamic, and other glandular, macrophagal, epithelial, stromal, and blastocoelic nature in which aberrant function, expression, activation or signaling of tyrosine kinases are involved.

Also provided herein is the use of a compound of Formula (I), (I-1), (I-2a), (I-2b), (I-2c), (I-3a), (I-3b), (I-3c), or a compound of Table 1, or a pharmaceutically acceptable salt thereof in the manufacture of a medicament for treatment of a proliferation disorder, such as a cancer, or a tumor in a subject.

In some embodiments, the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin (e.g., psoriasis) and benign hyperplasia of the prostate (e.g., BPH). In some embodiments, the compound of Formula (I), (I-1), (I-2a), (I-2b), (I-2c), (I-3a), (I-3b), (I-3c), or a compound of Table 1, may possess activity against brain metastases originated from these disorders.

Also provided are methods for inhibiting an activity of one or more kinases, such as MEK, COT1, FGFR4, MINK, MYO3A, PKG1B, and PLK3, which method comprises administering to an individual in need thereof a therapeutically effective amount of at least one chemical entity as described herein. In some embodiments, provided are methods of inhibiting one or more kinases, such as MEK, COT1, FGFR4, MINK, MYO3A, PKG1B, and PLK3 in a cell, comprising contacting the cell with at least one chemical entity as described herein, such as a compound of Formula (I), (I-1), (I-2a), (I-2b), (I-2c), (I-3a), (I-3b), (I-3c), or a compound of Table 1, or a pharmaceutically acceptable salt thereof. Additionally provided herein is the use of at least one chemical entity as described herein, such as a compound of Formula (I), (I-1), (I-2a), (I-2b), (I-2c), (I-3a), (I-3b), (I-3c), or a compound of Table 1, or a pharmaceutically acceptable salt thereof in the manufacture of a medicament for inhibiting an activity of one or more kinases, such as MEK, COT1, FGFR4, MINK, MYO3A, PKG1B, and PLK3 of an individual.

Also provided are methods for treating and/or preventing a proliferation disorder, such as a cancer, or a tumor in a subject which method comprises administering to an individual in need thereof a therapeutically effective amount of at least one chemical entity as described herein such as a compound of Formula (I), (I-1), (I-2a), (I-2b), (I-2c), (I-3a), (I-3b), (I-3c), or a compound of Table 1, or a pharmaceutically acceptable salt thereof. Additionally provided herein is the use of at least one chemical entity as described herein, such as a compound of Formula (I), (I-1), (I-2a), (I-2b), (I-2c), (I-3a), (I-3b), (I-3c), or a compound of Table 1, or a pharmaceutically acceptable salt thereof in the manufacture of a medicament for treating and/or preventing a proliferation disorder, a cancer, or a tumor in a subject.

In one embodiment, the disease or condition to be treated or prevented is abnormal cell proliferation such as cancer. The term “cancer” refers to pre-cancerous conditions, non-malignant, low-grade, high-grade, and malignant cancer. Cancer of any tissue type is contemplated for treatment or prevention by the compounds disclosed herein. Exemplary types of cancer include carcinoma, lymphoma, blastoma, sarcoma, leukemia, and lymphoid malignancies. More specifically, in certain embodiments the cancer is squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Provided herein is a method of treating cancer in an individual in need thereof by administering to the individual a therapeutically effective amount of a compound or composition described herein. Also provided herein is the use of a compound or composition described herein in the manufacture of a medicament for treatment of cancer in an individual in need thereof Also provided herein is the use of a compound or composition described herein for treatment of cancer in an individual in need thereof. Also provided herein is a compound or composition described herein for use in treatment of cancer in an individual in need thereof.

In another embodiment, the disease or condition to be treated or prevented is neurodegenerative disease. Exemplary types of neurodegenerative disease include, but are not limited to, Amyotrophic lateral sclerosis, Parkinson's disease, Alzheimer's disease, and Huntington's disease that occurs as a result of neurodegenerative processes.

In some embodiments, provided are methods of treating or preventing a neurodegenerative disease, such as Amyotrophic lateral sclerosis, Parkinson's disease, Alzheimer's disease, and Huntington's disease, comprising administering to the individual in need thereof a compound of Formula (I), (I-1), (I-2a), (I-2b), (I-2c), (I-3a), (I-3b), (I-3c), or a compound of Table 1, or a pharmaceutically acceptable salt thereof In some embodiments, provided are methods of treating or preventing a neurodegenerative disease, such as Amyotrophic lateral sclerosis, Parkinson's disease, Alzheimer's disease, and Huntington's disease, comprising administering to the subject a therapeutically effective amount of at least one chemical entity as described herein.

Provided herein is a method of treating a neurodegenerative disease in an individual in need thereof by administering to the individual a therapeutically effective amount of a compound or composition described herein. Also provided herein is the use of a compound or composition described herein in the manufacture of a medicament for treatment of a neurodegenerative disease in an individual in need thereof Also provided herein is the use of a compound or composition described herein for treatment of a neurodegenerative disease in an individual in need thereof. Also provided herein is a compound or composition described herein for use in treatment of neurodegenerative disease in an individual in need thereof.

In one aspect, provided herein are kits containing a compound or composition described herein and instructions for use. In some embodiments, the kits may contain instructions for use in the treatment of cancer in an individual in need thereof. In other embodiments, the kits may contain instructions for use in the treatment of a neurodegenerative disease in an individual in need thereof. A kit may additionally contain any materials or equipment that may be used in the administration of the compound or composition, such as vials, syringes, or IV bags. A kit may also contain sterile packaging.

### General Synthetic Methods

Compounds of Formula (I) will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate. Unless otherwise specified, the variables are as defined above in reference to Formula (I).

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

General methods of preparing compounds described herein are depicted in exemplified methods below. Variable groups in the schemes provided herein are defined as for Formula (I), (I-1), (I-2a), (I-2b), (I-2c), (I-3a), (I-3b), (I-3c), or any variation thereof. Other compounds described herein may be prepared by similar methods.

In some embodiments, the compound of Formula (I) is synthesized via the procedure as shown in Scheme A, wherein G¹, G², G³, G⁴, p , q, R¹, R², R³ and R⁴ are as defined for Formula (I), or any variation thereof detailed herein. Particular examples are provided in the Example section below.

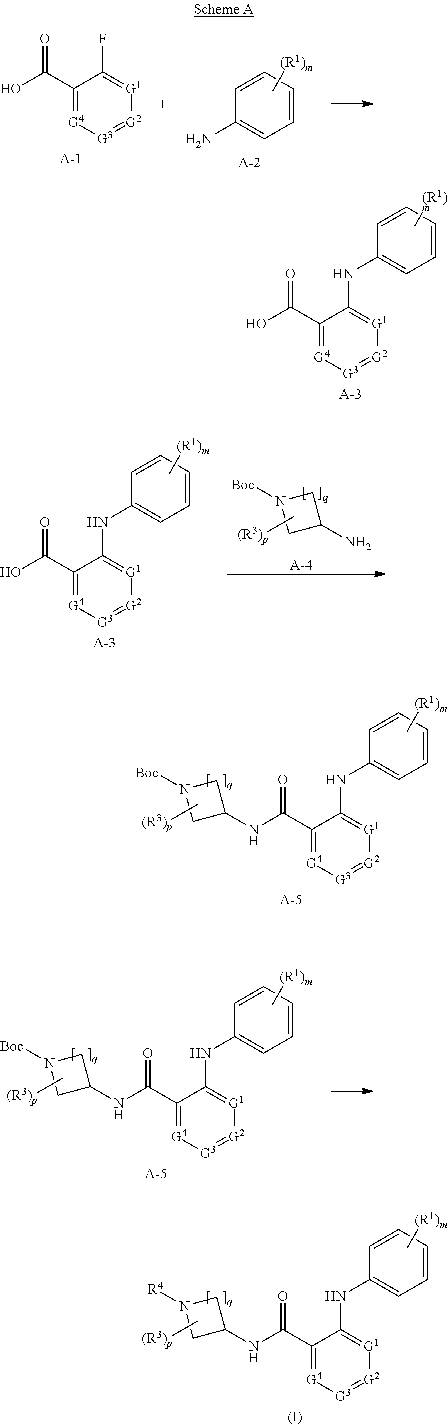

Starting materials, the synthesis of which is not specifically described above, are either commercially available or can be prepared using methods well known to those of skill in the art.

In some embodiments, the compound of Formula (I) is synthesized via the procedure as shown in Scheme B, wherein G¹ , G², G³, G⁴, p, q, R¹, R², R³, and R⁴ are as defined for Formula (I), or any variation thereof detailed herein. Particular examples are provided in the Example section below.

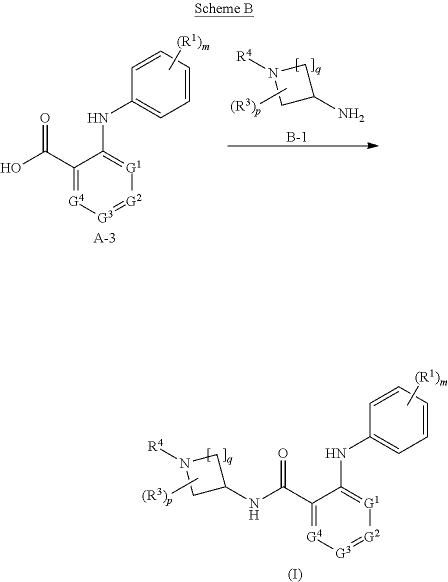

Starting materials, the synthesis of which is not specifically described above, are either commercially available or can be prepared using methods well known to those of skill in the art.

In some embodiments, the compound of Formula (I) is synthesized via the

procedure as shown in Scheme C, wherein G¹, G², G³, G⁴, p, q, R¹, R², R³, and R⁴ are as defined for Formula (I), or any variation thereof detailed herein, X is a leaving group suitable for a coupling reaction, such as halogen (e.g. bromo, iodo) for non-limiting example, and W-M is compound suitable for a coupling reaction, such as a boronic acid, a boronic acid ester, an organotin compound, an organozinc compound, an organosilicon compound, and a terminal alkyne, for non-limiting example. Particular examples are provided in the Example section below.

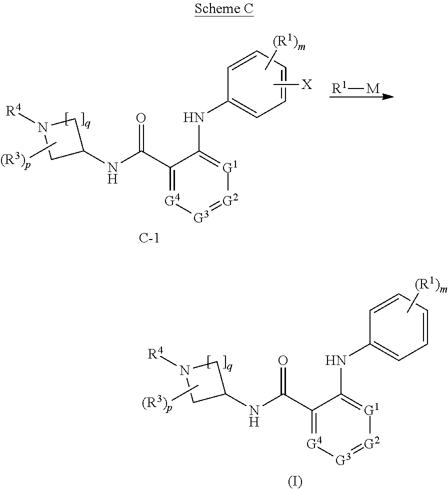

Starting materials, the synthesis of which is not specifically described above, are either commercially available or can be prepared using methods well known to those of skill in the art.

In some embodiments, the compound of Formula (I) is synthesized via the procedure as shown in Scheme D, wherein G¹, G², G³, G⁴, p, q, R¹, R², R³, and R⁴ are as defined for Formula (I), or any variation thereof detailed herein. Particular examples are provided in the Example section below.

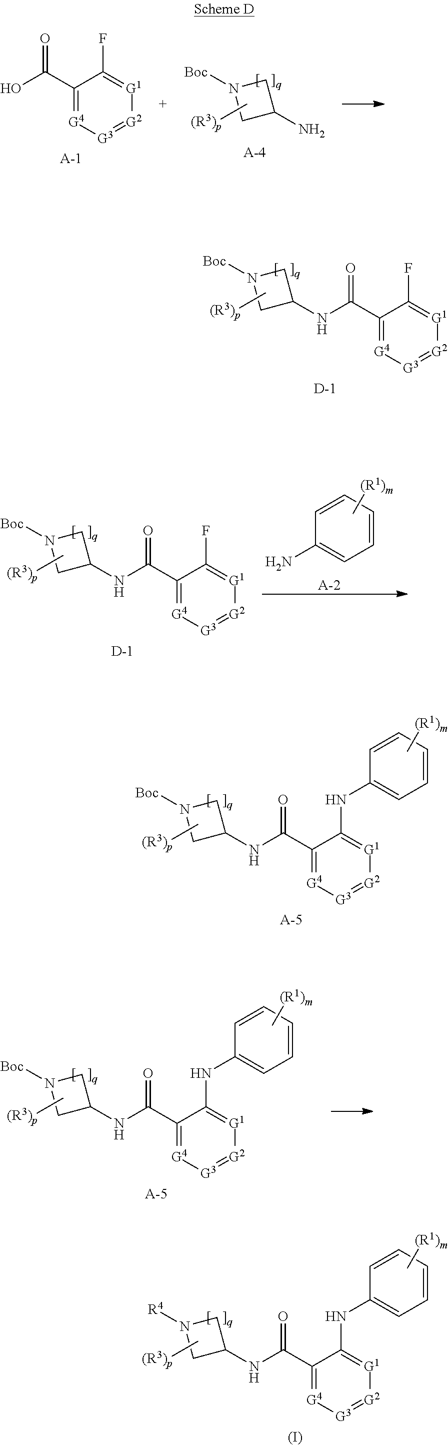

Starting materials, the synthesis of which is not specifically described above, are either commercially available or can be prepared using methods well known to those of skill in the art.

### EXAMPLES

The following examples are offered to illustrate but not to limit the compositions, uses, and methods provided herein. One of skill in the art will recognize that the following synthetic reactions and schemes may be modified by choice of suitable starting materials and reagents in order to access other compounds of Formula (I), (I-1), (I-2a), (I-2b), (I-2c), (I-3a), (I-3b), (I-3c), or a salt thereof. The compounds are prepared using the general methods described above.

The following chemical abbreviations are used throughout the Examples: ACN (acetonitrile), AcOH (acetic acid), CuI (copper(I) iodide), DCM (dichloromethane), DIEA (V, N-Diisopropylethylamine), DMF (dimethylformamide), DMSO (dimethyl sulfoxide), Et₃N (triethylamine), EtOAc (ethyl acetate), Et₂O (diethyl ether), ¹H NMR (proton nuclear magnetic resonance), HATU ((1-[bis(dimethylamino)methylene]-1H-1,2,3-triazolo[4,5-b]pyridinium 3-oxide hexafluorophosphate), HCl (hydrochloric acid), HPLC (high-performance liquid chromatography), K₂CO₃ (potassium carbonate), LCMS (Liquid chromatography—mass spectrometry), LiHMDS (lithium bis(trimethylsilyl)amide), LiNH₂ (lithium amide), MeOH (methanol), Na₂SO₄ (sodium sulfate), NaBH₃CN (sodium cyanoborohydride), NaOH (sodium hydroxide), n-BuLi (n-butyllithium), NH₃·H₂O (ammonia solution), PdCl₂(dppf) (1,1′-bis(diphenylphosphino)ferrocene palladium dichloride), PE (petroleum ether), prep-HPLC (preparative high-performance liquid chromatography), POCl₃ (phosphoryl chloride), TBAF (tetrabutylammonium fluoride), THF (tetrahydrofuran), and TFA (trifluoroacetic acid).

### Example 1: Preparation of N-(azetidin-3-yl)-2,4-difluoro-6-(2-fluoro-4-iodophenyl)amino)benzamide (Compound 1)

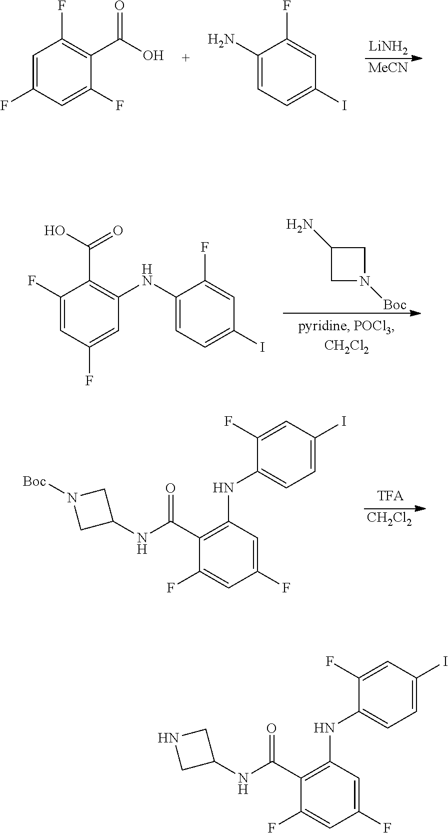

Step 1: Synthesis of 2,4-difluoro-6-((2-fluoro-4-iodophenyl)amino)benzoic acid

To a mixture of 2,4,6-trifluorobenzoic acid (646 mg, 3.67 mmol) and 2-fluoro-4-iodoaniline (1000 mg, 4.22 mmol) in MeCN (30 mL) was added LiNH₂ (295 mg, 12.8 mmol) under N₂. The reaction was stirred at 60° C. for 1 hour. After the reaction was completed, the reaction was cooled to room temperature, then 1N HCl was added until pH=2. The mixture was stirred for 30 minutes, then filtered. The resulting cake was dried to afford 2,4-difluoro-6-((2-fluoro-4-iodophenyl)amino)benzoic acid (1.22 g, 85% yield) as a pink solid.

Step 2: Synthesis of tent-butyl 3-(2,4-difluoro-6-((2-fluoro-4-iodophenyl)amino)benzamido)azetidine-1-carboxylate

To a solution of 2,4-difluoro-6-((2-fluoro-4-iodophenyl)amino)benzoic acid (200 mg, 0.51 mmol), tent-butyl 3-aminoazetidine-1-carboxylate (263 mg, 1.53 mmol) and pyridine (141 mg, 1.78 mmol) in CH₂Cl₂ (20 mL) was added POCl₃ (20 mg, 0.13 mmol). The reaction was stirred at room temperature for 16 hours. After the reaction was completed, the solvent was removed. The residue was purified by column chromatography on silica gel (PE/EtOAc=4/1) to afford tent-butyl 3-(2,4-difluoro-6-((2-fluoro-4-iodophenyl)amino)benzamido)azetidine-1-carboxylate (230 mg, 82% yield) as a white solid.

Step 3: Synthesis of N-(azetidin-3-yl)-2,4-difluoro-6-((2-fluoro-4-iodophenyl)amino)benzamide

To a solution of tent-butyl 3-(2,4-difluoro-6-((2-fluoro-4-iodophenyl)amino)benzamido)azetidine-1-carboxylate (170 mg, 0.31 mmol) in CH₂Cl₂ (10 mL) was added TFA (2 mL). The reaction was stirred at room temperature for 16 hours. After the reaction was completed, the solvent was removed, the residue was dissolved with CH₂Cl₂, NH₃·H₂O was added until pH>7, and the mixture was concentrated to dry. The residue was purified by prep-HPLC to afford N-(azetidin-3-yl)-2,4-difluoro-6-((2-fluoro-4-iodophenyl)amino)benzamide (TFA salt, 17 mg, 10% yield) as a yellow solid. ¹H NMR (400 MHz, DMSO-d₆): δ 9.23 (d, J=6.4 Hz, 1H), 8.77 (br s, 2H), 8.65 (s, 1H), 7.70 (dd, J=10.4, 1.6 Hz, 1H), 7.52 (d, J=8.4 Hz, 1H), 7.18 (t, J=8.4 Hz, 1H), 6.83-6.77 (m, 1H), 6.56 (d, J=11.2 Hz, 1H), 4.80-4.75 (m, 1H), 4.15 (t, J=9.2 Hz, 2H), 4.03 (t, J=9.2 Hz, 2H). LCMS (M+H⁺) m/z: 448.0.

### Example 2: Preparation of 2,4-difluoro-6-(2-fluoro-4-iodophenyl)amino)-N-(pyrrolidin-3-yl)benzamide (Compound 2)

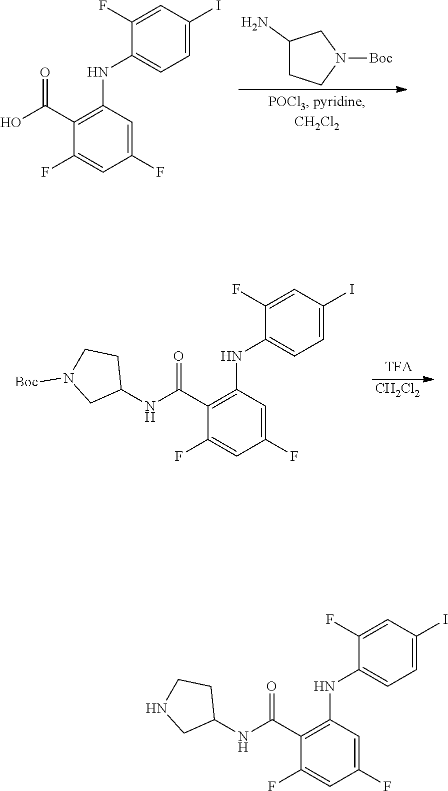 [SMILES: O=C(O)c1c(F)cc(F)cc1Nc1ccc(I)cc1F ; CC(C)(C)OC(=O)N1CCC(N)C1 ; CC(C)(C)OC(=O)N1CCC(NC(=O)c2c(F)cc(F)cc2Nc2ccc(I)cc2F)C1 ; "CH2Cl2" ; O=C(NC1CCNC1)c1c(F)cc(F)cc1Nc1ccc(I)cc1F]

Step 1: Synthesis of tent-butyl 3-(2,4-difluoro-6-((2-fluoro-4-iodophenyl)amino)benzamido)pyrrolidine-1-carboxylate

To a solution of 2,4-difluoro-6-((2-fluoro-4-iodophenyl)amino)benzoic acid (200 mg, 0.51 mmol), tent-butyl 3-aminopyrrolidine-1-carboxylate (285 mg, 1.53 mmol) and pyridine (141 mg, 1.78 mmol) in CH₂Cl₂ (20 mL) was added POCl₃ (20 mg, 0.13 mmol). The reaction was stirred at room temperature for 16 hours. After the reaction was completed, the solvent was removed. The residue was purified by column chromatography on silica gel (PE/EtOAc=4/1) to afford tent-butyl 3-(2,4-difluoro-6-((2-fluoro-4-iodophenyl)amino)benzamido)pyrrolidine-1-carboxylate (170 mg, 60% yield) as a white solid.

Step 2: Synthesis of 2,4-difluoro-6-((2-fluoro-4-iodophenyl)amino)-N-(pyrrolidin-3-yl)benzamide

To a solution of tent-butyl 3-(2,4-difluoro-6-((2-fluoro-4-iodophenyl)amino)benzamido)pyrrolidine-1-carboxylate (170 mg, 0.30 mmol) in CH₂Cl₂ (10 mL) was added TFA (2 mL). The reaction was stirred at room temperature for 16 hours. After the reaction was completed, the solvent was removed, the residue was dissolved with CH₂Cl₂, NH₃·H₂Owas added until pH>7, and the mixture was concentrated to dry. The residue was purified by column chromatography on silica gel (CH₂Cl₂/MeOH=25/1+0.5% NH₃·H₂O) to afford 2,4-difluoro-6-((2-fluoro-4-iodophenyl)amino)-N-(pyrrolidin-3-yl)benzamide (TFA salt, 130 mg, 75% yield) as a white solid. ¹H NMR (400 MHz, DMSO-d₆): δ 8.86-8.84 (m, 3H), 8.58 (s, 1H), 7.70 (dd, J=10.4, 2.0 Hz, 1H), 7.52 (d, J=8.4 Hz, 1H), 7.18 (t, J=8.4 Hz, 1H), 6.83-6.77 (m, 1H), 6.58 (d, J=10.4 Hz, 1H), 4.51-4.46 (m, 1H), 3.48-3.44 (m, 1H), 3.31-3.22 (m, 2H), 3.11-3.07 (m, 1H), 2.20-2.15 (m, 1H), 1.93-1.88 (m, 1H). LCMS (M+H⁺) m/z: 462.0.

### Example 3: Preparation of 2,4-difluoro-6-(2-fluoro-4-iodophenyl)amino)-N-(piperidin-3-yl)benzamide (Compound 3)

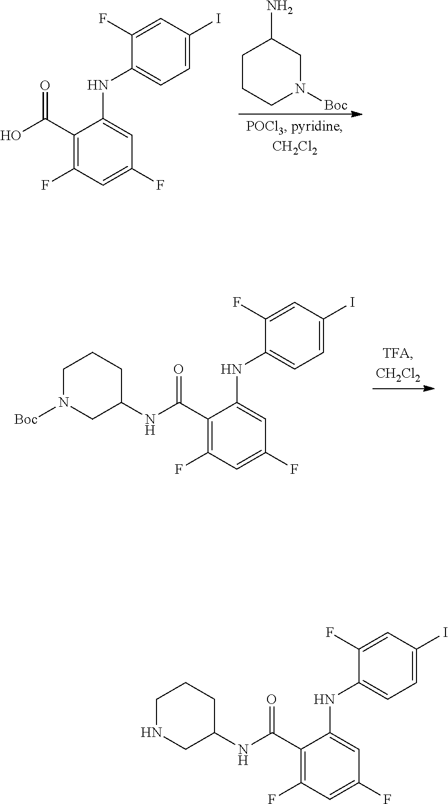

Step 1: Synthesis of tent-butyl 3-(2,4-difluoro-6-((2-fluoro-4-iodophenyl)amino)benzamido)piperidine-1-carboxylate

A solution of 2,4-difluoro-6-((2-fluoro-4-iodophenyl)amino)benzoic acid (200 mg, 0.50 mmol), tent-butyl 3-aminopiperidine-1-carboxylate (200 mg, 1.00 mmol), POCl₃ (3 drops) and pyridine (120 mg, 1.5 mmol) in CH₂Cl₂ (20 mL) was stirred at room temperature overnight. The reaction mixture was extracted with EtOAc, washed with brine, dried over Na₂SO₄ and concentrated, the residue was purified by column chromatography on silica gel (CH₂Cl₂/MeOH from 30:1 to 20:1, v/v) to afford tent-butyl 3-(2,4-difluoro-6-((2-fluoro-4-iodophenyl)amino)benzamido)piperidine-1-carboxylate (170 mg, 59% yield) as a white solid.

Step 2: Synthesis of 2,4-difluoro-6-((2-fluoro-4-iodophenyl)amino)-N-(piperidin-3-yl)benzamide

A mixture of tent-butyl 3-(2,4-difluoro-6-((2-fluoro-4-iodophenyl)amino)benzamido)piperidine-1-carboxylate (170 mg, 0.30 mmol) in CH₂Cl₂/TFA (10 mL/1 mL) was stirred at room temperature for 3 hours. After the reaction was completed, the solvent was evaporated and the residue was purified by prep-HPLC to afford 2,4-difluoro-6-((2-fluoro-4-iodophenyl)amino)-N-(piperidin-3-yl)benzamide (50 mg, 35% yield) as a white solid. ¹H NMR (600 MHz, DMSO-d₆): δ 8.67 (s, 1H), 8.36 (d, J=7.8 Hz, 1H), 7.68 (d, J=10.2 Hz, 1H), 7.49 (d, J=8.4 Hz, 1H), 7.20 (t, J=8.4 Hz, 1H), 6.75 (t, J=9.0 Hz, 1H), 6.60 (d, J=10.8 Hz, 1H), 3.79-3.77 (m, 1H), 2.91 (d, J=11.4 Hz, 1H), 2.73 (d, J=12.6 Hz, 1H), 2.45-2.38 (m, 2H), 1.81-1.79 (m, 1H), 1.61-1.59 (m, 1H), 1.43-1.37 (m, 2H). LCMS (M+H⁺) m/z: 476.0.

### Example 4: Preparation of N-(azetidin-3-yl)-2,3,4,5-tetrafluoro-6-(2-fluoro-4-iodophenyl)amino)benzamide (Compound 4)

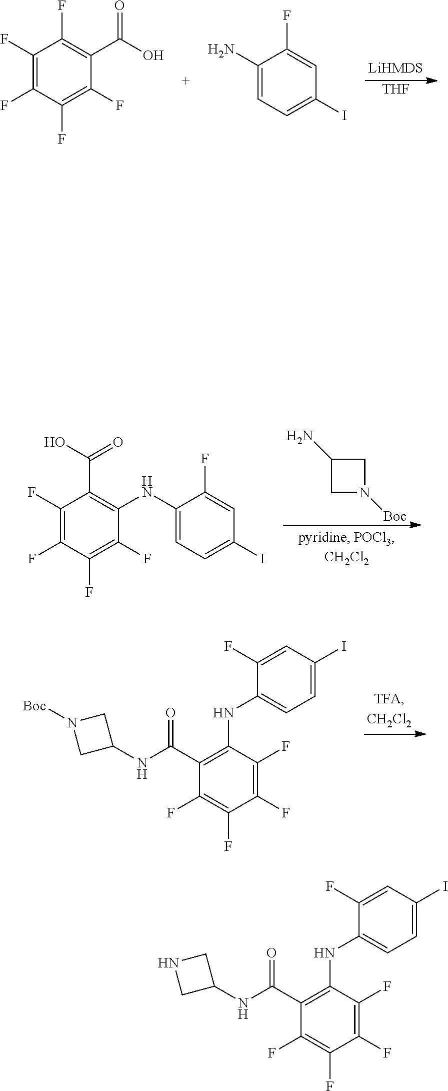

Step 1: Synthesis of 2,3,4,5-tetrafluoro-6-((2-fluoro-4-iodophenyl)amino)benzoic Acid

To a mixture of 2,3,4,5,6-pentafluorobenzoic acid (500 mg, 2.36 mmol) and 2-fluoro-4-iodoaniline (559 mg, 2.36 mmol) in THF (20 mL) was added LiHMDS (7 mL, 1 M in THF) under N₂, the reaction was stirred at room temperature for 16 hours. After the reaction was completed, the solvent was removed, the residue was purified by column chromatography on silica gel (CH₂Cl₂/MeOH=10/1) to afford 2,3,4,5-tetrafluoro-6-((2-fluoro-4-iodophenyl)amino)benzoic acid (700 mg, 69% yield) as a yellow solid.

Step 2: Synthesis of tent-butyl 3-(2,3,4,5-tetrafluoro-6-((2-fluoro-4-iodophenyl)amino)benzamido)azetidine-1-carboxylate

To a solution of 2,3,4,5-tetrafluoro-6-((2-fluoro-4-iodophenyl)amino)benzoic acid (200 mg, 0.47 mmol), tent-butyl 3-aminoazetidine-1-carboxylate (240 mg, 1.4 mmol) and pyridine (129 mg, 1.63 mmol) in CH₂Cl₂ (20 mL) was added POCl₃ (20 mg, 0.13 mmol). The reaction was stirred at room temperature for 16 hours. After the reaction was completed, the solvent was removed to give tent-butyl 3-(2,3,4,5-tetrafluoro-6-((2-fluoro-4-iodophenyl)amino)benzamido)azetidine-1-carboxylate (crude) which was used in the next step without purification.

Step 3: Synthesis of N-(azetidin-3-yl)-2,3,4,5-tetrafluoro-6-((2-fluoro-4-iodophenyl)amino)benzamide

To a solution of tent-butyl 3-(2,3,4,5-tetrafluoro-6-(2-fluoro-4-iodophenyl)amino)benzamido)azetidine-1-carboxylate (274 mg, 0.47 mmol) in CH₂Cl₂ (20 mL) was added TFA (2 mL). The reaction was stirred at room temperature for 2 hours. After the reaction was completed, the solvent was removed, the residue was dissolved with CH₂Cl₂, NH₃·H₂Owas added until pH>7, and the mixture was concentrated to dry. The residue was purified by column chromatography on silica gel (CH₂Cl₂/MeOH=25/1+0.5% NH₃·H₂O) to afford N-(azetidin-3-yl)-2,3,4,5-tetrafluoro-6-((2-fluoro-4-iodophenyl)amino)benzamide (73 mg, 32% yield for two steps) as a yellow solid. ¹H NMR (400 MHz, DMSO-d6): δ 9.45 (d, J=6.8 Hz, 1H), 8.76 (br s, 1H), 8.02 (s, 1H), 7.53 (dd, J=10.8, 1.6 Hz, 1H), 7.32 (d, J=8.4 Hz, 1H), 6.68-6.63 (m, 1H), 4.60-4.57 (m, 1H), 4.05-4.00 (m, 2H), 4.00-3.85 (m, 2H). LCMS (M+H⁺) m/z: 484.1.

### Example 5: Preparation of N-(azetidin-3-yl)-3-fluoro-5-((2-fluoro-4-iodophenyl)amino)isonicotinamide (Compound 5)

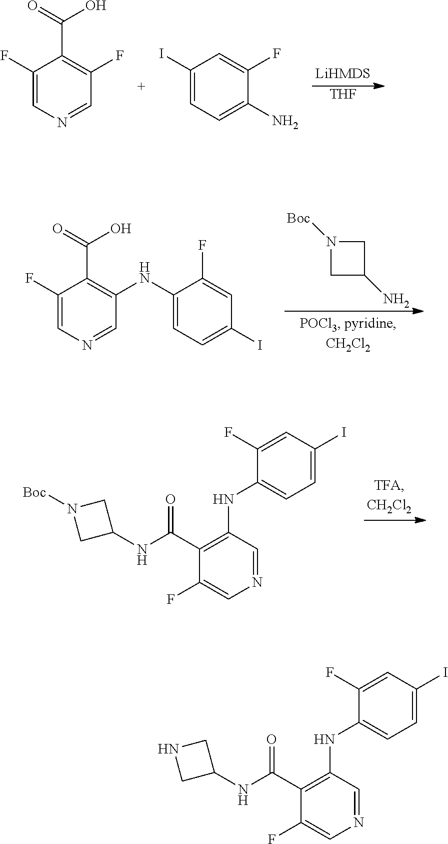

Step 1: Synthesis of 3-fluoro-5-((2-fluoro-4-iodophenyl)amino)isonicotinic acid

A mixture of 3,5-difluoroisonicotinic acid (5.00 g, 31.5 mmol) and 2-fluoro-4-iodoaniline (7.45 g, 31.5 mmol) in THF (200 mL) was cooled to 0° C. under N₂. LiHMDS (95 mL, 1 M in THF) was added, and the reaction was stirred at room temperature for 16 hours.

After the reaction was completed, the solvent was removed, the residue was dissolved with CH₂Cl₂ and washed with aqueous NaOH (2N, 500 mL). The aqueous layer was treated with concentrated HCl (100 mL) until pH=1, the mixture was filtered, and the cake was dried to afford 3-fluoro-5-((2-fluoro-4-iodophenyl)amino)isonicotinic acid (10 g, 85% yield) as a yellow solid.

Step 2: Synthesis of tent-butyl 3-(3-fluoro-5-((2-fluoro-4-iodophenyl)amino)isonicotinamido)azetidine-1-carboxylate

To a mixture of 3-fluoro-5-((2-fluoro-4-iodophenyl)amino)isonicotinic acid (10 g, 26.6 mmol) and tent-butyl 3-aminoazetidine-1-carboxylate (13.7 g, 79.8 mmol) in CH₂Cl₂ (300 mL) was added pyridine (7.4 g, 93 mmol) and POCl₃ (1 mL) under N₂. The reaction was stirred at room temperature for 16 hours. After the reaction was completed, the solvent was removed, and the residue was purified by column chromatography on silica gel (EtOAc 100%) to afford tent-butyl 3-(3-fluoro-5-((2-fluoro-4-iodophenyl)amino)isonicotinamido)azetidine-1-carboxylate (6.7 g, 48% yield).

Step 3: Synthesis of N-(azetidin-3-yl)-3-fluoro-5-((2-fluoro-4-iodophenyl)amino)isonicotinamide

To a solution of tent-butyl 3-(3-fluoro-5-((2-fluoro-4-iodophenyl)amino)isonicotinamido)azetidine-1-carboxylate (6.7 g, 12.6 mmol) in CH₂Cl₂ (200 mL) was added TFA (10 mL). The reaction was stirred at room temperature for 2 hours. After the reaction was completed, the solvent was removed. The residue was dissolved with CH₂Cl₂/MeOH (100 mL, 10/1), NH₃·H20 was added until pH>7, then the mixture was concentrated to dry, and the residue was treated with CH₂Cl₂ (100 mL). A yellow solid was formed after 30 minutes, filtered, and dried to afford N-(azetidin-3-yl)-3-fluoro-5-((2-fluoro-4-iodophenyl)amino)isonicotinamide (purity 100%, 4.13 g, 76% yield). The organic layer was concentrated to dry, and the residue was purified by column chromatography on silica gel (CH₂Cl₂/MeOH=10/1) to afford N-(azetidin-3-yl)-3-fluoro-5-((2-fluoro-4-iodophenyl)amino)isonicotinamide (contained some salts, 1.7 g, 31%) as a yellow solid. ¹H NMR (400 MHz, DMSO-d₆+D₂O): δ 8.18 (s, 1H), 8.13 (s, 1H), 7.67 (d, J=10.8 Hz, 1H), 7.49 (d, J=9.6 Hz, 1H), 7.08 (t, J=8.8 Hz, 1H), 4.74-4.66 (m, 1H), 4.14-4.09 (m, 2H), 4.01-3.96 (m, 2H). LCMS (M+H⁺) m/z: 431.2.

### Example 6: Preparation of N-(1-ethylazetidin-3-yl)-3-fluoro-5-((2-fluoro-4-iodophenyl)amino)isonicotinamide (Compound 6)

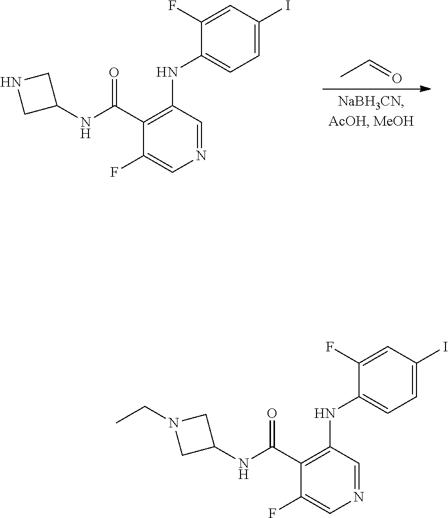

A mixture of N-(azetidin-3-yl)-3-fluoro-5-((2-fluoro-4-iodophenyl)amino)isonicotinamide (3.82 g, 8.88 mmol), AcOH (20 mg) and acetaldehyde (45 mL, 17.8 mmol) in MeOH (350 mL) was stirred at room temperature for 1 hour. NaBH₃CN (1.12 g, 17.8 mmol) was added, and the reaction was stirred at room temperature for 16 hours. After the reaction was completed, the solvent was removed. The residue was dissolved with CH₂Cl₂ (200 mL) and washed with water. The organic layer was dried and concentrated, and the residue was purified by column chromatography on silica gel (CH₂Cl₂/MeOH=25/1+0.5% NH₃·H₂O) to afford a yellow oil. Et₂O (50 mL) was added and the mixture was stirred for 30 minutes, filtered and dried to afford N-(1-ethylazetidin-3-yl)-3-fluoro-5-((2-fluoro-4-iodophenyl)amino)isonicotinamide (3 g, 74% yield) as a yellow solid. ¹H NMR (400 MHz, DMSO-d₆): δ 9.14 (d, J=7.2 Hz, 1H), 8.17 (s, 1H), 8.13 (s, 2H), 7.64 (dd, J=10.4, 1.6 Hz, 1H), 7.44 (d, J=8.4 Hz, 1H), 7.05 (t, J=8.4 Hz, 1H), 4.34-4.30 (m, 1H), 3.53 (t, J=7.2 Hz, 2H), 2.86 (t, J=6.4 Hz, 2H), 2.46-2.41 (m, 2H), 0.88 (t, J=7.2 Hz, 3H). LCMS (M+H⁺) m/z: 459.0.

### Example 7: Preparation of 3-fluoro-5-((2-fluoro-4-iodophenyl)amino)-N-(1-methylazetidin-3-yl)isonicotinamide (Compound 7)

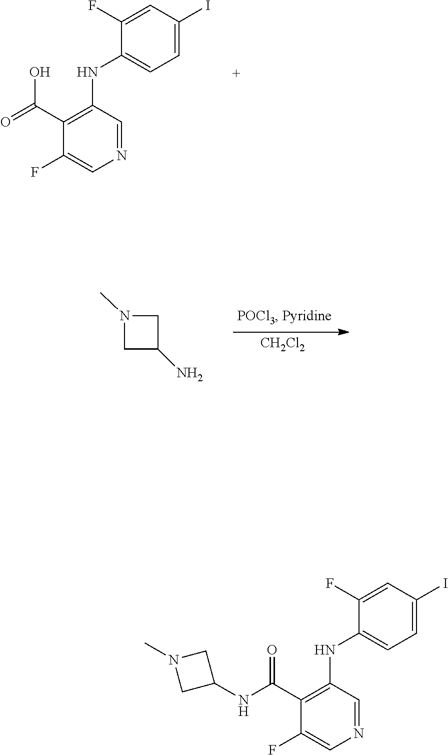

To a solution of 3-fluoro-5-((2-fluoro-4-iodophenyl)amino)isonicotinic acid (100 mg, 0.26 mmol) and 1-methylazetidin-3-amine (70 mg, 0.81 mmol) in CH₂Cl₂ (10 mL) was added pyridine (73 mg, 0.93 mmol) and POCl₃ (10 mg) under N₂, the reaction was stirred at room temperature for 16 hours. After the reaction was completed, the solvent was removed. The residue was purified by prep-HPLC to afford 3-fluoro-5-((2-fluoro-4-iodophenyl)amino)-N-(1-methylazetidin-3-yl)isonicotinamide (25 mg, 21% yield) as a yellow solid. ¹H NMR (400 MHz, DMSO-d₆): δ 9.16 (d, J=6.8 Hz, 1H), 8.18 (s, 1H), 8.14 (s, 2H), 7.65 (dd, J=10.4, 1.6 Hz, 1H), 7.45 (d, J=8.0 Hz, 1H), 7.06 (t, J=8.4 Hz, 1H), 4.32-4.26 (m, 1H), 3.51 (t, J=7.2 Hz, 2H), 2.84 (t, J=7.2 Hz, 2H), 2.23 (s, 3H). LCMS (M+H⁺) m/z: 445.1.

### Example 8: Preparation of 3-fluoro-5-((2-fluoro-4-iodophenyl)amino)-N-(1-isopropylazetidin-3-yl)isonicotinamide (Compound 8)

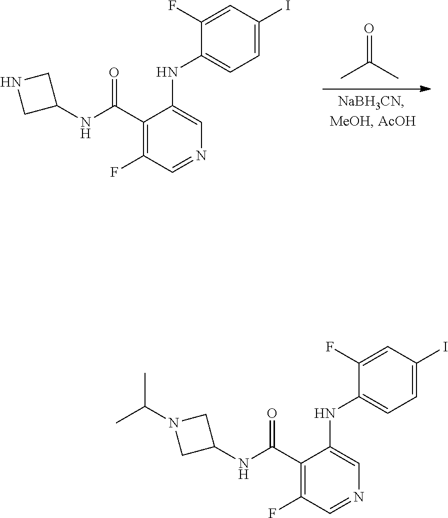

A solution of N-(azetidin-3-yl)-3-fluoro-5-((2-fluoro-4-iodophenyl)amino)isonicotinamide (215 mg, 0.50 mmol), acetone (1 mL), NaBH₃CN (63 mg, 1.0 mmol) and AcOH (3 drops) in MeOH (10 mL) was stirred at room temperature overnight. After the reaction was completed, the solvent was evaporated, and the residue was purified by prep-HPLC to afford 3-fluoro-5-((2-fluoro-4-iodophenyl)amino)-N-(1-isopropylazetidin-3-yl)isonicotinamide (50 mg, 21% yield) as a yellow solid. ¹H NMR (400 MHz, DMSO-d6): δ 9.09 (d, J=6.8 Hz 1H), 8.18 (s, 1H), 8.14-8.13 (m, 2H), 7.64 (dd, J=10.4, 2.0 Hz, 1H), 7.44 (dd, J=8.4, 1.2 Hz, 1H), 7.04 (t, J=8.4 Hz, 1H), 4.25-4.20 (m, 1H), 3.43 (t, J=7.2 Hz, 2H), 2.72 (t, J=7.2 Hz, 2H), 2.24-2.18 (m, 1H), 0.84 (d, J=6.0 Hz, 6H). LCMS (M+H⁺) m/z: 473.2.

### Example 9: Preparation of 3-fluoro-5-((2-fluoro-4-iodophenyl)amino)-N-(1-(2-fluoroethyl)azetidin-3-yl)isonicotinamide (Compound 9)

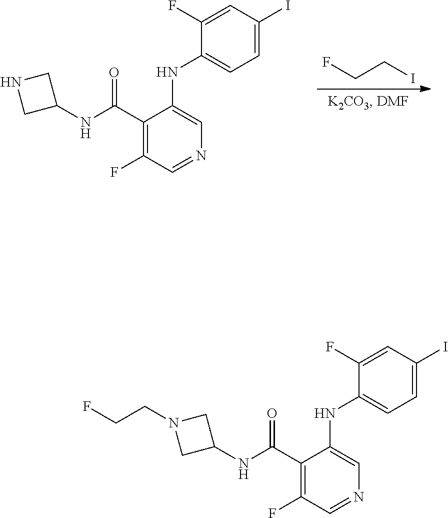

To a mixture of N-(azetidin-3-yl)-3-fluoro-5-((2-fluoro-4-iodophenyl)amino)isonicotinamide (130 mg, 0.3 mmol) and K₂CO₃ (82 mg, 0.6 mmol) in DMF (10 mL) was added 1-fluoro-2-iodoethane (42 mg, 0.24 mmol) at 0° C. Then the reaction was stirred at room temperature overnight. After removal of the solvent, the residue was dissolved in H₂O (20 mL). The mixture was extracted with EtOAc (50 mL×3) and dried over Na₂SO₄. The organic layer was concentrated and the residue was purified by prep-HPLC to give 3-fluoro-5-((2-fluoro-4-iodophenyl)amino)-N-(1-(2-fluoroethyl)azetidin-3-yl)isonicotinamide (10 mg, 7% yield) as a yellow solid. ¹H NMR (400 MHz, DMSO-d₆): δ 9.16 (d, J=6.8 Hz, 1H), 8.18 (s, 1H), 8.13 (s, 2H), 7.64 (d, J=10.8 Hz, 1H), 7.45 (d, J=8.4 Hz, 1H), 7.06 (t, J=8.4 Hz, 1H), 4.45 (t, J=4.8 Hz, 1H), 4.34-4.32 (m, 2H), 3.54 (t, J=6.8 Hz, 2H), 2.92 (t, J=6.8 Hz, 2H), 2.70 (t, J=4.8 Hz, 1H), 2.63 (t, J=4.8 Hz, 1H). LCMS (M+H⁺) m/z: 477.1.

### Example 10: Preparation of 3-fluoro-5-((2-fluoro-4-iodophenyl)amino)-N-(1-(2-methoxyethyl)azetidin-3-yl)isonicotinamide (Compound 10)

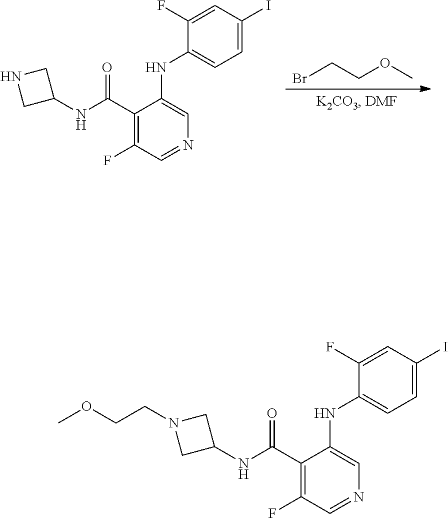

To a solution of N-(azetidin-3-yl)-3-fluoro-5-((2-fluoro-4-iodophenyl)amino)isonicotinamide (200 mg, 0.46 mmol) and K₂CO₃ (190 mg, 1.38 mmol) in DMF (5 mL) was added 1-bromo-2-methoxyethane (58 mg, 0.41 mmol) at 0° C. Then the reaction was stirred at room temperature overnight. After removal of the solvent, the residue was dissolved in H₂O (20 mL), the mixture was extracted with EtOAc (50 mL×3), dried over Na₂SO₄, the organic layer was concentrated. The residue was purified by prep-HPLC to give 3-fluoro-5-((2-fluoro-4-iodophenyl)amino)-N-(1-(2-methoxyethyl)azetidin-3-yl)isonicotinamide (38 mg, 17% yield) as a yellow solid. ¹H NMR (400 MHz, DMSO-d₆): δ 9.14 (d, J=7.2 Hz, 1H), 8.18 (s, 1H), 8.13-8.12 (m, 2H), 7.64 (dd, J=10.8, 2.0 Hz, 1H), 7.44 (dd, J=8.4, 0.8 Hz, 1H), 7.06 (t, J=8.8 Hz, 1H), 4.32-4.28 (m, 1H), 3.51-3.47 (m, 2H), 3.31-3.27 (m, 3H), 3.22 (s, 3H), 2.87-2.83 (m, 2H), 2.53-2.51 (m, 1H). LCMS (M+H⁺) m/z: 489.2.

### Example 11: Preparation of N-(azetidin-3-yl)-3-((4-ethynyl-2-fluorophenyl)amino)-5-fluoroisonicotinamide (Compound 11)

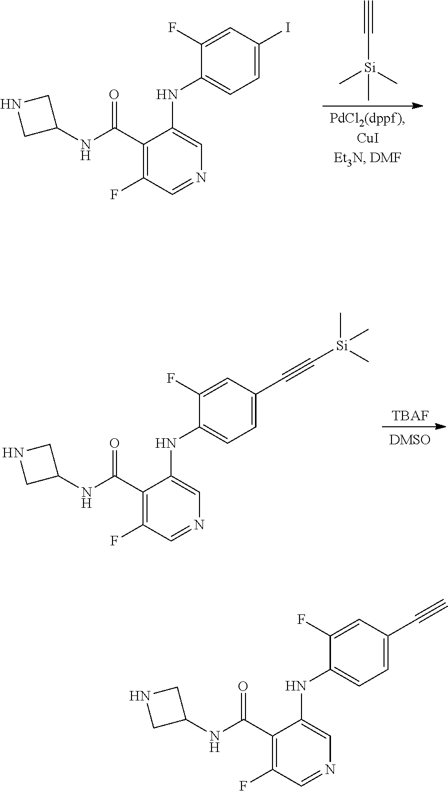

Step 1: Synthesis of N-(azetidin-3-yl)-3-fluoro-5-((2-fluoro-4-((trimethylsilyl)ethynyl)phenyl)amino)isonicotinamide

A mixture of N-(azetidin-3-yl)-3-fluoro-5-((2-fluoro-4-iodophenyl)amino)isonicotinamide (400 mg, 0.93 mmol) and ethynyltrimethylsilane (238 mg, 2.42 mmol) in DMF (10 mL) was added PdCl₂(dppf) (101 mg, 0.14 mmol) and CuI (50 mg, 0.28 mmol) and Et₃N (187 mg, 1.86 mmol) under N₂ at room temperature, the reaction was stirred at room temperature for 1 hour, the solvent was removed, the residue was dissolved with H₂O (100 mL), the mixture was extracted with CH₂Cl₂ (50 mL×3), dried over Na₂SO₄. The organic layer was concentrated to give N-(azetidin-3-yl)-3-fluoro-542-fluoro-4-((trimethylsilyl)ethynyl)phenyl)amino)isonicotinamide (150 mg, 40% yield) as a yellow solid.

Step 2: Synthesis of N-(azetidin-3-yl)-34(4-ethynyl-2-fluorophenyl)amino)-5-fluoroisonicotinamide

To a mixture of N-(azetidin-3-yl)-3-fluoro-5-((2-fluoro-4-((trimethylsilyl)ethynyl)phenyl)amino)isonicotinamide (153 mg, 0.38 mmol) in DMSO (2 mL) was added TBAF (1 M, 0.38 mL) and the reaction mixture was stirred at room temperature for 30 minutes. The reaction was concentrated, and the crude residue was purified by prep-HPLC to give N-(azetidin-3-yl)-3-((4-ethynyl-2-fluorophenyl)amino)-5-fluoroisonicotinamide (TFA salt, 40 mg, 24% yield) as a yellow solid. ¹H NMR (400 MHz, DMSO-d₆): δ 9.48 (d, J=6.4 Hz, 1H), 8.77 (br s, 2H), 8.39 (s, 1H), 8.26 (s, 1H), 8.24 (s, 1H), 7.39 (d, J=10.8 Hz, 1H), 7.25-7.21 (m, 2H), 4.74-4.67 (m, 1H), 4.23 (s, 1H), 4.14-4.09 (m, 2H), 4.00-3.95 (m, 2H). LCMS (M+H⁺) m/z: 329.2.

### Example 12: Preparation of N-(1-ethylazetidin-3-yl)-3-((4-ethynyl-2-fluorophenyl)amino)-5-fluoroisonicotinamide (Compound 12)

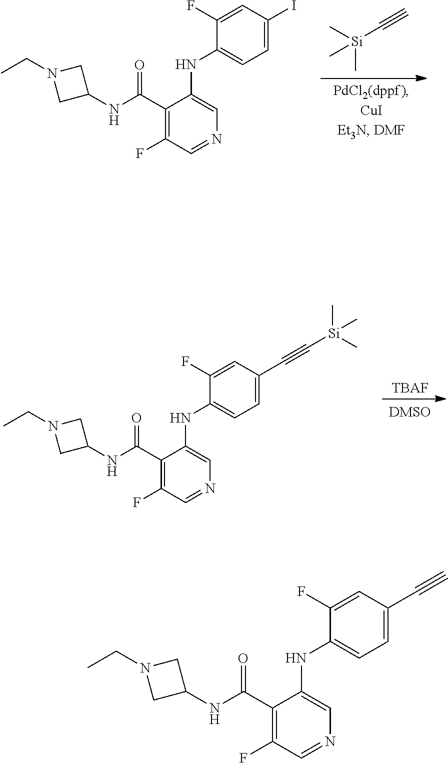

Step 1: Synthesis of N-(1-ethylazetidin-3-yl)-3-fluoro-5-((2-fluoro-4-((trimethylsilyl)ethynyl)phenyl)amino)isonicotinamide

To a solution of N-(1-ethylazetidin-3-yl)-3-fluoro-5-((2-fluoro-4-iodophenyl)amino)isonicotinamide (400 mg, 0.87 mmol) and ethynyltrimethylsilane (255 mg, 2.61 mmol) in DMF (10 mL) was added PdCl₂(dppf) (94 mg, 0.13 mmol) and CuI (50 mg, 0.26 mmol) and Et₃N (175mg, 1.74 mmol) under N₂ at room temperature. The reaction mixture was stirred at room temperature for 2 hours, the solvent was removed, the residue was dissolved in H₂O (100 mL), the mixture was extracted with CH₂Cl₂ (50 mL×3), dried over Na₂SO₄. The organic layer was concentrated and the residue was purified by column chromatography on silica gel (CH₂Cl₂/MeOH=10/1) to give N-(1-ethylazetidin-3-yl)-3-fluoro-5-((2-fluoro-4-((trimethylsilyl)ethynyl)phenyl)amino)isonicotinamide (340 mg, 91% yield) as a yellow solid.

Step 2: Synthesis of N-(1-ethylazetidin-3-yl)-3-((4-ethynyl-2-fluorophenyl)amino)-5-fluoroisonicotinamide

To a solution of N-(1-ethyl azetidin-3-yl)-3-fluoro-5-((2-fluoro-4-((trimethylsilyl)ethynyl)phenyl)amino)isonicotinamide (340 mg, 0.79 mmol) in DMSO (2 mL) was added TBAF (1 M, 0.79 mL) at room temperature for 30 minutes, the reaction was concentrated to give the crude, the residue was purified by prep-HPLC to give N-(1-ethyl azetidin-3-yl)-3 ((4-ethynyl-2-fluorophenyl)amino)-5-fluoroisonicotinamide (TFA salt, 27 mg, 7% yield) as a yellow solid. ¹H NMR (400 MHz, DMSO-d₆): δ 10.10-10.06 (m, 1H), 9.56-9.47 (m, 1H), 8.46-8.38 (m, 1H), 8.26-8.24 (m, 2H), 7.45-7.39 (m, 1H), 7.27-7.25 (m, 2H), 4.70-4.65 (m, 1H), 4.42-4.37 (m, 1H), 4.22-4.19 (m, 2H), 3.90-3.86 (m, 1H), 3.25-3.17 (m, 2H), 1.11-1.06 (m, 3H). LCMS (M+H⁺) m/z: 357.3.

### Example 13: Preparation of N-(azetidin-3-yl)-3-chloro-5-((2-fluoro-4-iodophenyl)amino)isonicotinamide (Compound 13)

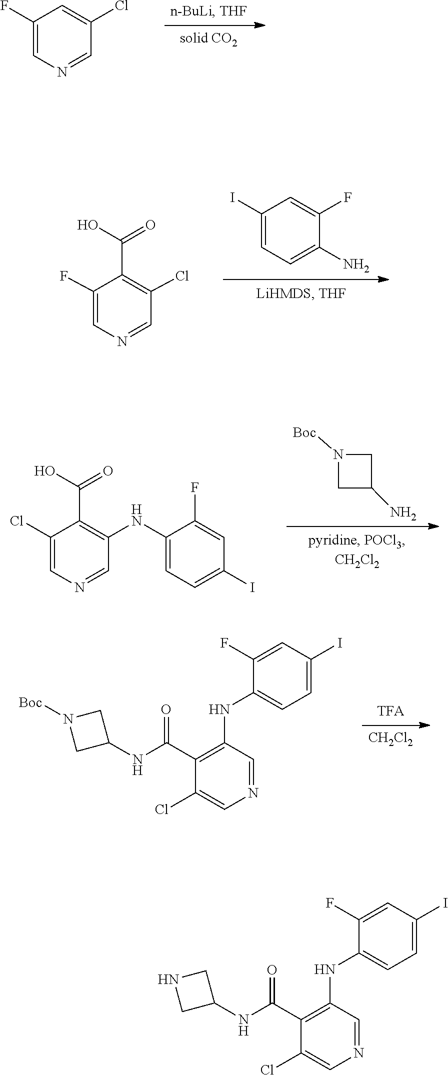

Step 1: Synthesis of 3-chloro-5-fluoroisonicotinic Acid

To a solution of 3-chloro-5-fluoropyridine (500 mg, 3.8 mmol) in THF (20 mL) cooled to −78° C. under N₂, was added n-BuLi (4.75 mL, 1.6 M in hexane). The reaction was stirred at −78° C. for 1 hour, solid CO₂ was added, and the reaction was stirred at room temperature for 16 hours. The reaction was not completed (90% of desired compound and 8% of started material). The solvent was removed, and the residue was washed with CH₂Cl₂, filtered and dried to afford 3-chloro-5-fluoroisonicotinic acid (670 mg, crude) as a white solid, which was used to the next step directly without purification.

Step 2: Synthesis of 3-chloro-5-((2-fluoro-4-iodophenyl)amino)isonicotinic Acid

A mixture of 3-chloro-5-fluoroisonicotinic acid (670 mg, 3.8 mmol) and 2-fluoro-4-iodoaniline (900 mg, 3.8 mmol) in THF (30 mL) was cooled to 0° C. under N₂. LiHMDS (11.4 mL, 1 M in THF) was added, and the reaction was stirred at room temperature for 16 hours. After the reaction was completed, the solvent was removed, the residue was washed with CH₂Cl₂ and 2N NaOH, the combined aqueous layers were treated with concentrated HCl until pH=1, and the mixture was stirred at room temperature for 16 hours. The mixture was filtered and dried to afford 3-chloro-5-((2-fluoro-4-iodophenyl)amino)isonicotinic acid (240 mg, 16% yield for 2 steps) as a yellow solid.

Step 3: Synthesis of tent-butyl 3-(3-chloro-5-((2-fluoro-4-iodophenyl)amino)isonicotinamido)azetidine-1-carboxylate

To a solution of 3-chloro-5-((2-fluoro-4-iodophenyl)amino)isonicotinic acid (240 mg, 0.61 mmol) and tent-butyl 3-aminoazetidine-1-carboxylate (316 mg, 1.84 mmol) in CH₂Cl₂ (20 mL) was added pyridine (170 mg, 2.14 mmol) and POCl₃ (30 mg) under N₂, and the reaction was stirred at room temperature for 16 hours. After the reaction was completed, the solvent was removed to give tent-butyl 3-(3-chloro-5-((2-fluoro-4-iodophenyl)amino)isonicotinamido)azetidine-1-carboxylate which was used to the next step without purification.

Step 4: Synthesis of N-(azetidin-3-yl)-3-chloro-5-((2-fluoro-4-iodophenyl)amino)isonicotinamide

To a solution of tent-butyl 3-(3-chloro-5-((2-fluoro-4-iodophenyl)amino)isonicotinamido)azetidine-1-carboxylate (330 mg, 0.60 mmol) in CH₂Cl₂ (30 mL) was added TFA (3 mL). The reaction was stirred at room temperature for 2 hours. After the reaction was completed, the solvent was removed. The residue was purified by column chromatography on silica gel (CH₂Cl₂/MeOH=25/1+0.5% NH₃·H₂O) to afford N-(azetidin-3-yl)-3-chloro-5-((2-fluoro-4-iodophenyl)amino)isonicotinamide (180 mg, 66% yield for 2 steps) as a yellow solid. ¹H NMR (400 MHz, DMSO-d₆): δ 9.18 (d, J=7.2 Hz, 1H), 8.22 (s, 1H), 8.12 (s, 1H), 7.74 (s, 1H), 7.61 (dd, J=10.4, 1.6 Hz, 1H), 7.43 (d, J=8.0 Hz, 1H), 6.97 (t, J=8.4 Hz, 1H), 4.56-4.52 (m, 1H), 3.57 (t, J=7.6 Hz, 2H), 3.46 (t, J=7.6 Hz, 2H). LCMS (M+H⁺) m/z: 447.1.

### Example 14: Preparation of N-(Azetidin-3-yl)-3-((4-bromo-2-chlorophenyl)amino)-5-fluoroisonicotinamide (Compound 14)

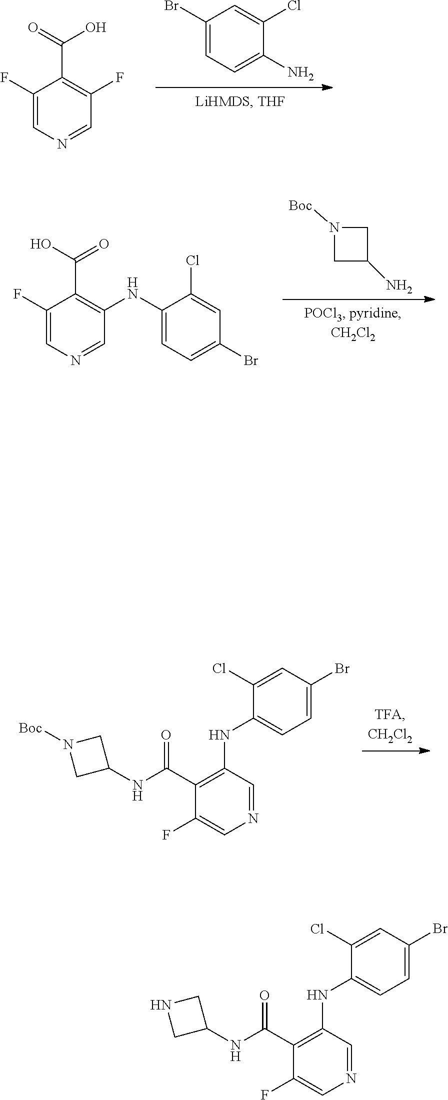 [SMILES: O=C(O)c1c(F)cncc1F ; Nc1ccc(Br)cc1Cl ; O=C(O)c1c(F)cncc1Nc1ccc(Br)cc1Cl ; CC(C)(C)OC(=O)N1CC(N)C1 ; CC(C)(C)OC(=O)N1CC(NC(=O)c2c(F)cncc2Nc2ccc(Br)cc2Cl)C1 ; O=C(NC1CNC1)c1c(F)cncc1Nc1ccc(Br)cc1Cl]

Step 1: Synthesis of 3((4-bromo-2-chlorophenyl)amino)-5-fluoroisonicotinic Acid

A mixture of 3,5-difluoroisonicotinic acid (160 mg, 1.0 mmol) and 4-bromo-2-chloroaniline (207 mg, 1.0 mmol) in THF (10 mL) was cooled to 0° C. under N₂. LiHMDS (3 mL, 1 M in THF) was added, and the reaction was stirred at room temperature for 16 hours. After the reaction was completed, the solvent was removed, and the residue was purified by column chromatography on silica gel (CH₂Cl₂/MeOH=10/1) to afford 3-((4-bromo-2-chlorophenyl)amino)-5-fluoroisonicotinic acid (180 mg, 52% yield) as a yellow solid.

Step 2: Synthesis of tent-butyl 3-(3-((4-bromo-2-chlorophenyl)amino)-5-fluoroisonicotinamido)azetidine-1-carboxylate

To a solution of 3-((4-bromo-2-chlorophenyl)amino)-5-fluoroisonicotinic acid (180 mg, 0.52 mmol) and tent-butyl 3-aminoazetidine-1-carboxylate (269 mg, 1.56 mmol) in CH₂Cl₂ (20 mL) was added pyridine (144 mg, 1.83 mmol) and POCl₃ (25 mg) under N₂, and the reaction was stirred at room temperature for 16 hours. After the reaction was completed, the solvent was removed to give tent-butyl 3-(3-((4-bromo-2-chlorophenyl)amino)-5-fluoroisonicotinamido)azetidine-1-carboxylate (crude) which was used to the next step without purification.

Step 3: Synthesis of N-(azetidin-3-yl)-3-((4-bromo-2-chlorophenyl)amino)-5-fluoroisonicotinamide

To a solution of tent-butyl 3-(3-((4-bromo-2-chlorophenyl)amino)-5-fluoroisonicotinamido)azetidine-1-carboxylate (260 mg, 0.52 mmol) in CH₂Cl₂ (30 mL) was added TFA (3 mL), and the reaction was stirred at room temperature for 2 hours. After the reaction was completed, the solvent was removed. The residue was purified by column chromatography on silica gel (CH₂Cl₂/MeOH=15/1+0.5% NH₃·H₂O) to afford N-(azetidin-3-yl)-3-((4-bromo-2-chlorophenyl)amino)-5-fluoroisonicotinamide (120 mg, 58% yield for 2 steps) as a yellow solid. 1 1-1 NMR (400 MHz, DMSO-d₆₊D20): δ 8.30 (s, 1H), 8.25 (s, 1H), 7.73 (d, J=2.4 Hz, 1H), 7.43 (dd, J=8.8, 2.4 Hz, 1H), 7.24 (d, J=8.4 Hz, 1H), 4.61-4.56 (m, 1H), 3.58-3.54 (m, 2H), 3.47-3.45 (m, 2H). LCMS (M+H⁺) m/z: 399.1.

### Example 15: Preparation of N-(azetidin-3-yl)-3-((2-fluoro-4-iodophenyl)amino)isonicotinamide (Compound 15)

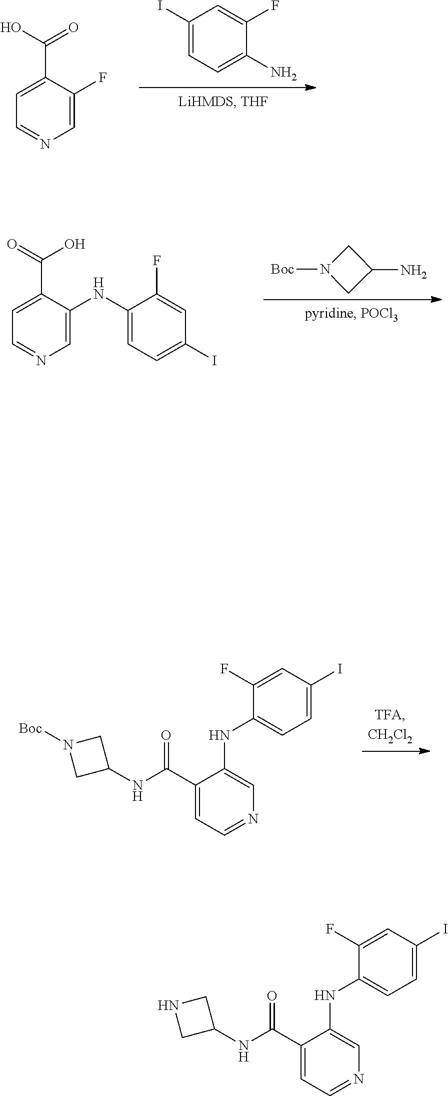 [SMILES: O=C(O)c1ccncc1F ; Nc1ccc(I)cc1F ; O=C(O)c1ccncc1Nc1ccc(I)cc1F ; CC(C)(C)OC(=O)N1CC(N)C1 ; CC(C)(C)OC(=O)N1CC(NC(=O)c2ccncc2Nc2ccc(I)cc2F)C1 ; O=C(NC1CNC1)c1ccncc1Nc1ccc(I)cc1F]

Step 1: Synthesis of 3-((2-fluoro-4-iodophenyl)amino)isonicotinic acid

To a solution of 3-fluoroisonicotinic acid (820 mg, 5.8 mmol) and 2-fluoro-4-iodoaniline (1380 mg, 5.8 mmol) in THF (30 mL) was added LiHMDS (17.4 mL, 17.4 mmol) at 0° C. The reaction mixture was stirred at room temperature overnight. To the mixture was added 1N aqueous NaOH (20 mL). The mixture was extracted by EtOAc (50 mL×2). The water phase was acidified by addition 1N HCl to pH=5-7, the mixture was stirred for 30 minutes and then filtered, and the cake was dried to afford 3-((2-fluoro-4-iodophenyl)amino)isonicotinic acid (350 mg, 17% yield) as a white solid.

Step 2: Synthesis of tent-butyl 3-(3-((2-fluoro-4-iodophenyl)amino)isonicotinamido)azetidine-1-carboxylate

To a mixture of 3-((2-fluoro-4-iodophenyl)amino)isonicotinic acid (200 mg, 0.55 mmol) and tent-butyl 3-aminoazetidine-1-carboxylate (290 mg, 1.68 mmol) in pyridine (10 mL) was added POCl₃ (6 drops). The reaction mixture was stirred at room temperature overnight. The reaction mixture was concentrated, and the residue was purified by column chromatography on silica gel (CH₂Cl₂/MeOH from 100:1 to 30:1, v/v) to afford tent-butyl 3-(3-((2-fluoro-4-iodophenyl)amino)isonicotinamido)azetidine-1-carboxylate (100 mg, 35% yield) as a brown solid.

Step 3: Synthesis of N-(azetidin-3-yl)-3-((2-fluoro-4-iodophenyl)amino)isonicotinamide

A mixture of tent-butyl 3-(3-((2-fluoro-4-iodophenyl)amino)isonicotinamido)azetidine-1-carboxylate (100 mg, 0.19 mmol) in CH₂Cl₂/TFA (10 mL/1 mL) was stirred at room temperature for 3 hours. After the reaction was completed, the solvent was evaporate to afford N-(azetidin-3-yl)-3-((2-fluoro-4-iodophenyl)amino)isonicotinamide (TFA salt, 100 mg, 98% yield) as brown oil. ¹H NMR (400 MHz, DMSO-d₆): δ 9.53 (d, J=6.4 Hz, 1H), 9.20 (s, 1H), 8.85 (br s, 2H), 8.51 (s, 1H), 8.25 (d, J=4.8 Hz, 1H), 7.72-7.67 (m, 2H), 7.50 (dd, J=8.4, 0.8 Hz, 1H), 7.26 (t, J=8.4 Hz, 1H), 4.81-4.75 (m, 1H), 4.17-4.08 (m, 4H). LCMS (M+H⁺) m/z: 413.0.

### Example 16: Preparation of N-(azetidin-3-yl)-4-((2-fluoro-4-iodophenyl)amino)nicotinamide (Compound 16)

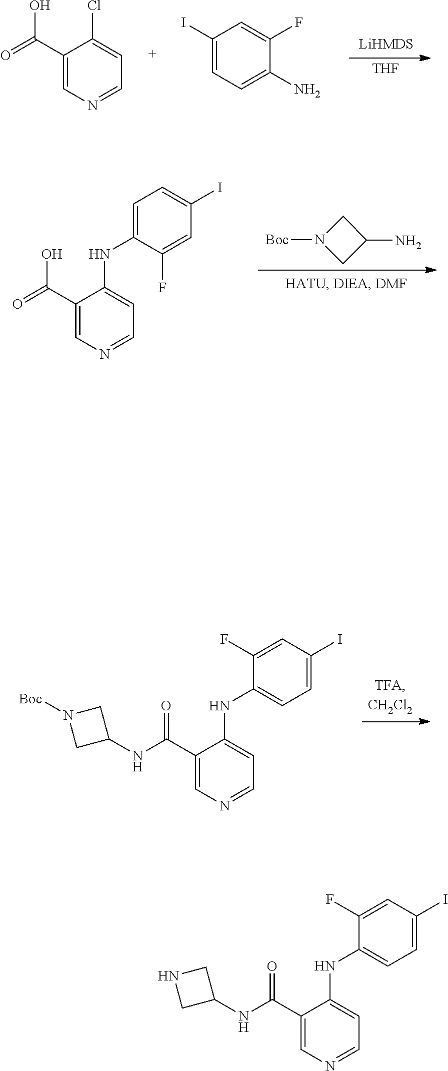

Step 1: Synthesis of 4-((2-fluoro-4-iodophenyl)amino)nicotinic Acid

To a solution of 4-chloronicotinic acid (1.0 g, 6.3 mmol) and 2-fluoro-4-iodoaniline (1.5 g, 6.3 mmol) in THF (40 mL) was added LiHMDS (19.1 mL, 19.1 mmol) at 0° C. The reaction mixture was stirred at room temperature overnight. To the mixture was added 1N aqueous NaOH (20 mL). The mixture was extracted by EtOAc (60 mL×2). The water phase was acidified by addition of 1N HCl to pH=5-7, the mixture was stirred for 30 minutes and then filtered, and the cake was dried to afford 4-((2-fluoro-4-iodophenyl)amino)nicotinic acid (1.0 g, 45% yield) as a white solid.

Step 2: Synthesis of tent-butyl 3-(4-((2-fluoro-4-iodophenyl)amino)nicotinamido)azetidine-1-carboxylate

A mixture of 4-((2-fluoro-4-iodophenyl)amino)nicotinic acid (200 mg, 0.55 mmol), tent-butyl 3-aminoazetidine-1-carboxylate (193 mg, 1.12 mmol), HATU (319 mg, 0.84 mmol) and DIEA (145 mg, 1.12 mmol) in DMF (10 mL) was stirred at room temperature overnight. The reaction mixture was concentrated, the residue was purified by prep-HPLC to afford tent-butyl 3-(4-((2-fluoro-4-iodophenyl)amino)nicotinamido)azetidine-1-carboxylate (180 mg, 63% yield) as a white solid.

Step 3: Synthesis of N-(azetidin-3-yl)-4-((2-fluoro-4-iodophenyl)amino)nicotinamide

A mixture of tent-butyl 3-(4-((2-fluoro-4-iodophenyl)amino)nicotinamido)azetidine-1-carboxylate (180 mg, 0.35 mmol) in CH₂Cl₂/TFA (15 mL/1.5 mL) was stirred at room temperature for 3 hours. After the reaction was completed, the solvent was evaporated to afford N-(azetidin-3-yl)-4-((2-fluoro-4-iodophenyl)amino)nicotinamide (TFA salt, 80 mg, 43% yield) as brown oil. ¹H NMR (400 MHz, DMSO-d₆): δ 10.66 (s, 1H), 9.80 (d, J=6.0 Hz, 1H), 9.09 (br s, 2H), 8.88 (s, 1H), 8.37 (d, J=7.6 Hz, 1H), 7.90 (dd, J=10.0, 2.0 Hz, 1H), 7.71 (dd, J=8.4, 1.2 Hz, 1H), 7.31 (t, J=8.4 Hz, 1H), 6.98 (dd, J=6.8, 2.0 Hz, 1H), 4.86-4.83 (m, 1H), 4.21-4.14 (m, 4H). LCMS (M+H⁺) m/z: 413.1.

### Example 17: Preparation of 3-((4-acetyl-2-fluorophenyl)amino)-N-(azetidin-3-yl)-5-fluoroisonicotinamide (Compound 17)

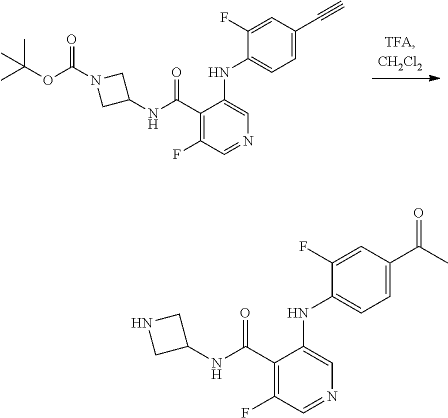 [SMILES: C#Cc1ccc(Nc2cncc(F)c2C(=O)NC2CN(C(=O)OC(C)(C)C)C2)c(F)c1 ; CC(=O)c1ccc(Nc2cncc(F)c2C(=O)NC2CNC2)c(F)c1]

Step 1: Synthesis of 3-((4-acetyl-2-fluorophenyl)amino)-N-(azetidin-3-yl)-5-fluoroisonicotinamide

To a solution of tent-butyl 3-(3-((4-ethynyl-2-fluorophenyl)amino)-5-fluoroisonicotinamido)azetidine-1-carboxylate (500 mg, 1.2 mmol) in DCM (10 mL), TFA (1 mL) was added at room temperature. The mixture was stirred at room temperature for 3 hours. Concentrated to give the crude, then purified by prep-HPLC (0.1% TFA/MeCN/H₂O) to afford 3-((4-acetyl-2-fluorophenyl)amino)-N-(azetidin-3-yl)-5-fluoroisonicotinamide (5 mg, yield 1%, TFA salt) as a yellow solid. ¹H NMR (400 MHz, DMSO-d₆): δ 9.48 (d, J=6.8 Hz, 1H), 8.70-8.67 (m, 3H), 8.40 (d, J=5.2 Hz, 2H), 7.77 (dd, J=8.4, 2.0 Hz, 1H), 7.70 (dd, J=8.4, 2.0 Hz, 1H), 7.23 (t, J=8.4 Hz, 1H), 4.68-4.66 (m, 1H), 4.10-4.06 (m, 2H), 3.95-3.90 (m, 2H), 2.54 (s, 3H). LCMS (M+H⁺) m/z: 347.3.

### Example 18: Preparation of 3-(3-(3-((2-fluoro-4-iodophenyl)amino)isonicotinamido)azetidin-1-yl)propanoic Acid (Compound 18)

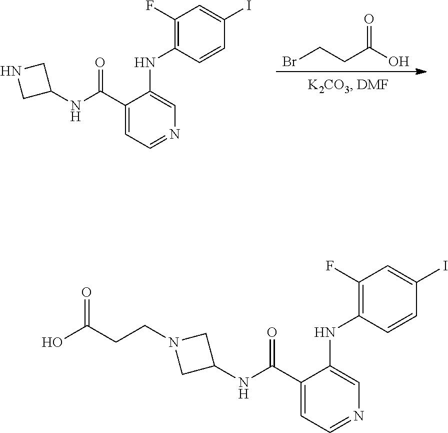 [SMILES: O=C(NC1CNC1)c1ccncc1Nc1ccc(I)cc1F ; O=C(O)CCBr ; O=C(O)CCN1CC(NC(=O)c2ccncc2Nc2ccc(I)cc2F)C1]

Step 1: Synthesis of 3-(3-(3-((2-fluoro-4-iodophenyl)amino)isonicotinamido)azetidin-1-yl)propanoic Acid

A mixture of N-(azetidin-3-yl)-3-((2-fluoro-4-iodophenyl)amino)isonicotinamide (32 mg, 0.06 mmol), K₂CO₃ (17 mg, 0.12 mmol) and 3-bromopropanoic acid (10 mg, 0.06 mmol) in DMF (5 mL) was stirred at room temperature for 16 hours. After the reaction was completed, the solvent was removed, the residue was purified by prep-HPLC (0.1% TFA/MeCN/H₂O) to afford 3-(3-(3-((2-fluoro-4-iodophenyl)amino)isonicotinamido)azetidin-1-yl)propanoic acid (12 mg, 40% yield, TFA salt) as a yellow solid. ¹H NMR (400 MHz, DMSO-d₆+D₂O): δ 8.50 (s, 1H), 8.25 (d, J=4.0 Hz, 1H), 7.69 (dd, J=10.4, 1.6 Hz, 1H), 7.65 (s, 1H), 7.51 (d, J=8.4 Hz, 1H), 7.25 (t, J=8.4 Hz, 1H), 4.74-4.68 (m, 1H), 4.46-4.42 (m, 2H), 4.12-4.08 (m, 2H), 3.45-3.41 (m, 2H), 2.62 (t, J=6.8 Hz, 2H). LCMS (M+H⁺) m/z: 485.2.

### Example 19: Preparation of 4-(3-(3-((2-fluoro-4-iodophenyl)amino)isonicotinamido)azetidin-1-yl)butanoic acid (Compound 19)

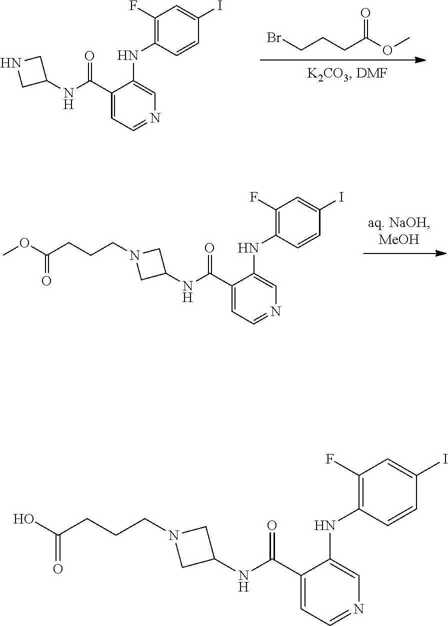

Step 1: Synthesis of methyl 4-(3-(3-((2-fluoro-4-iodophenyl)amino)isonicotinamido)azetidin-1-yl)butanoate

A mixture of N-(azetidin-3-yl)-342-fluoro-4-iodophenyl)amino)isonicotinamide (100 mg, 0.24 mmol), methyl 4-bromobutanoate (43 mg, 0.24 mmol) and K₂CO₃ (67 mg, 0.49 mmol) in DMF (5 mL) was stirred at room temperature for 16 hours. After the reaction was completed, the solvent was removed, and the residue was used to the next step without purification.

Step 2: Synthesis of 4-(3-(3-((2-fluoro-4-iodophenyl)amino)isonicotinamido)azetidin-1-yl)butanoic Acid

To a solution of methyl 4-(3-(3-((2-fluoro-4-iodophenyl)amino)isonicotinamido)azetidin-1-yl)butanoate (100 mg, 0.19 mmol) in MeOH (5 mL) was added aqueous NaOH (2N, 5 mL), and the reaction mixture was stirred at room temperature for 1 hour. After the reaction was completed, the mixture was concentrated and the residue was purified by prep-HPLC (0.1% TFA/MeCN/H₂O) to afford 4-(3-(3-((2-fluoro-4-iodophenyl)amino)isonicotinamido)azetidin-1-yl)butanoic acid (100 mg, 86% yield, TFA salt) as a yellow solid. ¹H NMR (400 MHz, DMSO-d₆+D₂O): δ 8.50 (s, 1H), 8.25 (s, 1H), 7.72-7.65 (m, 2H), 7.52 (d, J=8.4 Hz, 1H), 7.25 (t, J=8.4 Hz, 1H), 4.77-4.72 (m, 1H), 4.50-4.45 (m, 1H), 4.37-4.33 (m, 1H), 4.27-4.22 (m, 1H), 4.07-4.03 (m, 1H), 3.24-3.20 (m, 2H), 2.34 (t, J=7.2 Hz, 2H), 1.72-1.69 (m, 2H). LCMS (M+H⁺) m/z: 499.2.

### Example 20: Preparation of N-(1-acetylazetidin-3-yl)-3-fluoro-54(2-fluoro-4-iodophenyl)amino)isonicotinamide (Compound 20)

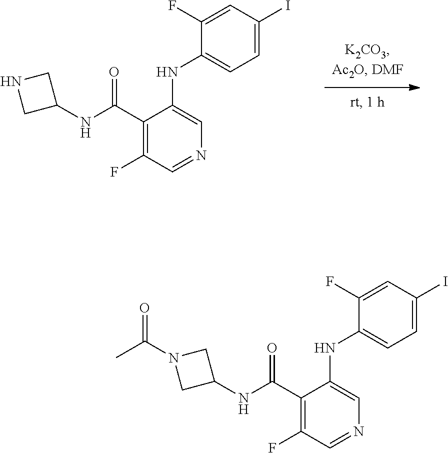 [SMILES: O=C(NC1CNC1)c1c(F)cncc1Nc1ccc(I)cc1F ; CC(=O)N1CC(NC(=O)c2c(F)cncc2Nc2ccc(I)cc2F)C1]

Step 1: Synthesis of N-(1-acetylazetidin-3-yl)-3-fluoro-5-((2-fluoro-4-iodophenyl)amino)isonicotinamide

To a solution of N-(azetidin-3-yl)-3-fluoro-5-((2-fluoro-4-iodophenyl)amino)isonicotinamide (60 mg, 0.14 mmol) and acetic anhydride (21 mg, 0.21 mmol) in DMF (1 mL) was added K₂CO₃ (39 mg, 0.28 mmol), and the mixture was stirred at room temperature for 1 hour. The reaction mixture was filtered and purified by Prep-HPLC (0.1% NH₃-H₂O) to afford N-(1-acetylazetidin-3-yl)-3-fluoro-5-((2-fluoro-4-iodophenyl)amino)isonicotinamide (34 mg, 52% yield) as a yellow solid. ¹H NMR (400 MHz, DMSO-d₆): δ 9.37 (s, 1H), 8.21-8.13 (m, 3H), 7.66 (dd, J=10.8, 2.0 Hz, 1H), 7.47 (dd, J=8.4, 1.2 Hz, 1H), 7.07 (t, J=8.4 Hz, 1H), 4.54-4.50 (m, 1H), 4.35 (t, J=8.4 Hz, 1H), 4.07 (t, J=8.4 Hz, 1H), 3.88-3.84 (m, 1H), 3.74-3.70 (m, 1H), 1.76 (s, 3H). LCMS (M+H⁺) m/z: 473.0.

While the foregoing written description of the compounds, uses, and methods described herein enables one of ordinary skill to make and use the compounds, uses, and methods described herein, those of ordinary skill will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

All references disclosed herein are incorporated by reference in their entirety.

The in vitro and in vivo activities of the compounds of Formula (I) were determined using the following procedures.

### Biological Example B1

### MEKI/2 Inhibition Assay

The test compounds were dissolved in 100% DMSO to prepare 10 mM stock solution. For test compounds, 100× solution with 4-fold serial dilution and a total of 7 concentrations were prepared. Staurosporine, as positive control compound used 100× solution with 3-fold serial dilution and a total of 10 concentrations. The final starting concentrations for test compounds and Staurosporine were 2 μM and 0.1 respectively. With automated liquid handler, 250 nL compounds were transferred to 384 well plate according to plate map. And MEK1 (Carna, 07-141) to 2.5 ×final concentration (0.015 nM) were diluted with 1×Kinase buffer containing 40 nM of inactive ERK2 (Carna, 04-143-10). Add 10 uL enzyme mix was added for each well in 384 well plate. 10 uL 1×Kinase buffer was used as the negative control. The enzyme mix and compounds were pre-incubated at RT for 10 minutes. The 15 μL of substrate mix containing ATP (100 μM) and Kinase substrate 8 (GL BioChem, 112396) were added into 384 well plate, and react at RT for 30 min. 30 μL stop buffer were added to stop reaction. Read conversion rate with Caliper EZ Reader.

The compound inhibition rate is calculated as:

% Inhibition=

(Conversion %_max−Conversion %_sample)/(Conversion %_max−Conversion %_min)×100
Conversion %_sample: Conversion % value of the sample
Conversion %_min: Average conversion % value of negative control
Conversion %_max: Average conversion % value of positive control.

The dose-response curve is fitted with GraphPad Prism Sand IC₅₀ calculated bylog(inhibitor) vs. response—Variable slopeprogram with the following formula:

Y=Bottom+(Top−Bottom)/(1+10\hat{ }((Log IC₅₀−X)*HillSlope))

### Antiproliferation Assays

In vitro anti-proliferation study of test compounds in A375 cell line by CellTiter Glo. The cells were routinely maintained as a monolayer culture in corresponding culture medium, at 37° C. with 5% CO₂ in air.

Exponential growth cells were harvested by trypsin-EDTA digestion. Cell pellet was re-suspended in fresh culture medium, and the concentration adjusted as needed (the cell density per well was listed in following form). The cell viability was over 98% by Trypan blue staining. Cells were incoluated into 96 wells plates according to the plate map (90 μL/well). Plates were incubated at 37° C. and 5% CO₂ overnight. The next day, the 10×compound containing medium was prepared according to the plate map. 10 μL of 10×compound containing medium was transferred into each well of the assay plates (the final DMSO concentration was 0.5%). The medium was gently mixed and incubated at 37° C. and 5% CO₂ for another 72 hours or 144 hours.

Reagent was prepared according to the manufacture's instruction. 50 μL CellTiter-Glo Reagent were added in each well. Contents were mixed for 2 minutes on an orbital shaker to induce cell lysis. The plate was incubated at room temperature for 10 minutes to stabilize luminescent signal. 100 μL of reaction contents were transferred of each well from the clear plates into white walled/white opaque 96-well plates. Luminescence was recorded on Envision.

Using the luminescence measurements [time zero (T0), control growth (C), and test growth in the presence of drug at the six concentration levels (Ti)], the percentage growth was calculated at each of the drug concentrations levels.

Percentage growth inhibition (GI) was calculated as: GI (%)=[(Ti−T0)/(C-T0)]×100 for concentrations for which Ti>=T0, and GI (%)=[(Ti−T0)/T0]×100 for concentrations for which Ti<T0. Data were analyzed using the XLFit (Excel) tool, fitting to a 4-parameter equation to generate concentration response curves. Concentration of compound that inhibits 50% of control cell growth (GI50) was back-interpolated when y=50% of net growth of DMSO treated control wells using nonlinear regression with the equation: f(x) 205 [fit=(A+((B−A)/(1+((C/x)\hat{ }D))))], where A is the minimum response (Ymin), B is the maximum response (Ymax), C is the inflection point of the curve (Re GI50) and D is the Hill coefficient. Growth inhibition of 50% (GI50) was calculated at the 50% Growth inhibition on the curve. Index values were the sum of Inhibition rate (IR) at each tested compound concentration.

Table A shows the antiproliferation of synthesized compounds at A375 melanoma cells and HT-29 colon cancer cells.

TABLE A

| Compound | A375 | HT-29 |
|---|---|---|
| No. | GI50 | GI50 |
| 1 | ++ | + |
| 2 | + | N.D. |
| 3 | + | N.D. |
| 4 | ++ | + |
| 5 | +++ | +++ |
| 6 | +++ | +++ |
| 7 | +++ | N.D. |
| 8 | N.D. | N.D. |
| 9 | N.D. | +++ |
| 10 | N.D. | |
| 11 | +++ | +++ |
| 12 | ++ | ++ |
| 13 | +++ | N.D. |
| 14 | ++ | + |
| 15 | N.D. | +++ |
| 16 | N.D. | N.D. |
| 17 | N.D. | N.D. |
| 18 | + | N.D. |
| 19 | ++ | N.D. |
| 20 | + | N.D. |

+++: < 100 nM;
++: 100-1,000 nM;
+: >1,000 nM;
N.D.: Not Determined

### Biological Example B2

### MDCK-MDR1 Permeability Assay

MDCK-MDR1 cells originated from transfection of Madin Darby canine kidney (MDCK) cells with the MDR1 gene, the gene encoding for the efflux protein, P-glycoprotein. This cell line is ideal for identifying substrates of P-gp, with or without an inhibitor. The cells were seeded on a Multiscreen™ plate to form a confluent monolayer over 4 days prior to the experiment. On day 4, the test compound (1-30 μM concentration) was added to the apical side of the membrane and the transport of the compound across the monolayer was monitored over a 120 minutes time period. To study drug efflux, it was also necessary to investigate transport of the compound from the basolateral compartment to the apical compartment and calculate an efflux ratio.

The permeability coefficient (Pₐₚₚ) was calculated from the following equation:

Pₐₚₚ=[(dQ/dt)/C₀×A]

where dQ/dt is the rate of permeation of the drug across the cells, C₀ is the donor compartment concentration at time zero and A is the area of the cell monolayer.

An efflux ratio was calculated from the mean apical to basolateral (A−B) Pₐₚₚ data and basolateral to apical (B−A) Pₐₚₚ data.

Efflux Ratio=Pₐₚₚ(B−A)/Pₐₚₚ(A−B)

Table B summarizes the permeability of Compound 1 in a MDCK-MDR1 Assay.

TABLE B

| Cmpd No. | Papp (a to b) | Papp (b to a) | Efflux Ratio |
|---|---|---|---|
| 5 | 10.54 | 36.01 | 3.42 |
| 6 | 34.10 | 18.00 | 0.53 |
| 7 | 32.46 | 18.98 | 0.58 |
| 11 | 14.59 | 59.50 | 4.08 |
| 13 | 4.30 | 50.80 | 11.81 |

### Biological Example B3

### Caco-2 Permeability Assay

Caco-2 cells are widely used as an in vitro assay to measure the permeability of a drug compound. The Caco-2 cell line is derived from a human colorectal carcinoma, and when cultured, the cells spontaneously differentiate into monolayers of polarized enterocytes. Caco-2 cells express P-glycoprotein and breast cancer resistance protein, two of the most relevant cell membrane active transporters that affect drug compound's permeability into cells and blood brain barrier.

The cells were seeded on Millipore Millicell plates and formed a confluent monolayer over 20 days prior to the experiment. On day 20, the test compound (1-30 uM concentration) was added to the apical side of the membrane and the transport of the compound across the monolayer was monitored over a 120 minutes time period. To study drug efflux, it was also necessary to investigate transport of the compound from the basolateral compartment to the apical compartment.

The permeability coefficient (Pₐₚₚ) was calculated from the following equation:

Pₐₚₚ=[(dQ/dt)/C₀×A]

where dQ/dt is the rate of permeation of the drug across the cells, C₀ is the donor compartment concentration at time zero and A is the area of the cell monolayer. C₀ is obtained from analysis of the dosing solution at the start experiment.

The permeability of selected compounds in the Caco-2 Assay is summarized in Table C.

TABLE C

| Cmpd No. | Papp (a to b) | Papp (b to a) | Efflux Ratio |
|---|---|---|---|
| 6 | 89.32 | 42.28 | 0.47 |
| 11 | 8.79 | 20.47 | 2.33 |

### Biological Example B4

### Mouse Pharmacokinetics Study

The pharmacokinetic properties of Compounds 5 and 6 was studied in CD-1 mice via intravenous and oral administration by using a standard protocol. The test articles were formulated in 20% Hydroxypropyl-beta-cyclodextrin, either as a clear solution or fine suspension. Table D shows the pharmacokinetic characterization by intravenous injection of Compounds 5 and 6 in mice.

TABLE D

| | IV Dose | | Clₚ | V_{d} |
|---|---|---|---|---|
| Cmpd No. | (mg/kg) | t_{1/2} (h) | (ml/min · kg) | (L/kg) |
| 6 | 5 | 0.625 ± 0.043 | 77.8 ± 8.0 | 4.20 ± 0.33 |
| 11 | 5 | 9.03 ± 0.69 | 86.0 ± 12.0 | 67.7 ± 13.9 |

Table E shows the plasma exposure by oral administration of Compound 1 in mice.

TABLE E

| | PO Dose | Cₘₐₓ | Tₘₐₓ | AUC | F |
|---|---|---|---|---|---|
| Cmpd No. | (mg/kg) | (ng/ml) | (h) | (ng/mL · h) | (%) |
| 6 | 25 | 1074 ± 328 | 0.50 ± 0.00 | 3157 ± 641 | 62.1 ± 11.4 |
| 11 | 25 | 1700 ± 95 | 0.67 ± 0.29 | 4546 ± 301 | 93.1 ± 6.0 |

### Biological Example B5

### In Vivo Pharmacodynamic Study

The activity of the compounds of formula (I), in vivo, can be determined by the amount of inhibition of tumor growth by a test compound relative to a control. The tumor growth inhibitory effects of various compounds are measured according to the method of Corbett T. H., et al., “Tumor Induction Relationships in Development of Transplantable Cancers of the Colon in Mice for Chemotherapy Assays, with a Note on Carcinogen Structure”, Cancer Res., 35, 2434-2439 (1975) and Corbett T. H., et al., “A Mouse Colon-tumor Model for Experimental Therapy”, Cancer Chemother. Rep. (Part 2)”, 5, 169-186 (1975), with slight modifications. Tumors are induced in the left flank by subcutaneous injection of 1-5 million log phase cultured tumor cells (human A375 melanoma or HT-29 colorectal cancer cells) suspended in 0.1 ml RPMI 1640 medium. After sufficient time has elapsed for the tumors to become palpable (100-150 mm³ in size/5-6 mm in diameter) the test animals (BALB/c nude female mice) are treated with test compound (formulated at a concentration of 10 to 15 mg/ml in 20% hydroxypropyl-beta-cyclodextrine) by oral route of administration once or twice daily. In order to determine an anti-tumor effect, the tumor is measured in millimeters with a Vernier caliper across two diameters and the tumor size (mm3) is calculated using the formula: Tumor size (mm³)=(length×width)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula: Inhibition (%)=(TuW_{control}−TuWₜₑₛₜ)/TuW_{control}×100%. The flank site of tumor implantation provides reproducible dose/response effects for a variety of chemotherapeutic agents, and the method of measurement (tumor diameter) is a reliable method for assessing tumor growth rates.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

1. A compound of Formula (I): 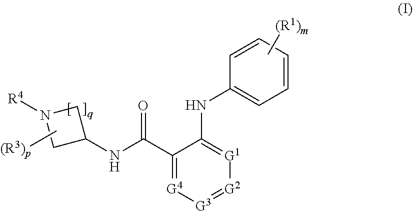
or a pharmaceutically acceptable salt, solvate or isotopic derivative thereof, wherein:
G¹ is CH or CR²
G², G³, and G⁴ are independently of each other N, CH, or CR²;
provided that at least two of G², G³, and G⁴ are independently of each other CH or CR²;
and further provided that when G¹ is CH, then at least one of G², G³, and G⁴ is N or CR²;
m is 0, 1, 2, 3, 4, or 5;
p is 0, 1, 2, 3, or 4;
q is 1, 2, or 3;
each R¹ is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl);
each R² is independently selected from the group consisting of halogen, OH, NH₂, NO₂, CN, optionally substituted C₁-C₆ alkyl, optionally substituted C₂-C₆ alkenyl, optionally substituted C₂-C₆ alkynyl, optionally substituted C₁-C₆ haloalkyl, optionally substituted C₁-C₆ alkoxy, -[(optionally substituted C₁-C₆ alkylene)-(optionally substituted C₁-C₆ alkoxy)], —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl);
each R³ is independently selected from the group consisting of halogen, OH, NH₂, optionally substituted C₁-C₆ alkyl, optionally substituted C₁-C₆ haloalkyl, -[(optionally substituted C₁-C₆ alkylene)-(optionally substituted C₁-C₆ alkoxy)], optionally substituted C₁-C₆ alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl); and
R⁴ is selected from the group consisting of hydrogen, halogen, OH, NH₂, optionally substituted C₁-C₆ alkyl, optionally substituted C₁-C₆ haloalkyl, -[(optionally substituted C₁-C₆ alkylene)-(optionally substituted C₁-C₆ alkoxy)], optionally substituted C₁-C₆ alkylene-COOH, optionally substituted C₁-C₆ alkylene-C(═O)—O-(optionally substituted C₁-C₆ alkyl), optionally substituted C₁-C₆ alkoxy, —O-[(optionally substituted C₁-C₆ alkylene)-(optionally substituted C₁-C₆ alkoxy)], —NH(optionally substituted C₁-C₆ alkyl), —N(optionally substituted C₁-C₆ alkyl)₂, —NH[(optionally substituted C₁-C₆ alkylene)-(optionally substituted C₁-C₆ alkoxy)], —N[(optionally substituted C₁-C₆ alkylene)-(optionally substituted C₁-C₆ alkoxy)]₂, —C(═O)-(optionally substituted C₁-C₆ alkyl), —C(═O)-(optionally substituted C₂-C₆ alkenyl), and —C(═O)-(optionally substituted C₂-C₆ alkynyl).

2. The compound of claim 1, or a pharmaceutically acceptable salt, solvate or isotopic derivative thereof, wherein the compound of Formula (I) is a compound of Formula (I-1): 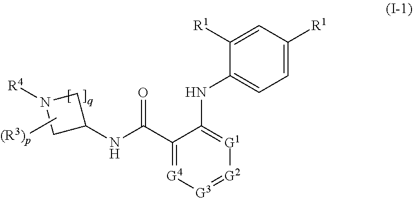
or a pharmaceutically acceptable salt, solvate or isotopic derivative thereof, wherein G¹, G², G³, G⁴, p, q, R¹, R², R³, and R⁴ are as defined for Formula (I).

3. The compound of claim 1, or a pharmaceutically acceptable salt, solvate or isotopic derivative thereof, wherein the compound of Formula (I) is a compound of Formula (I-2a), (I-2b), or (I-2c): 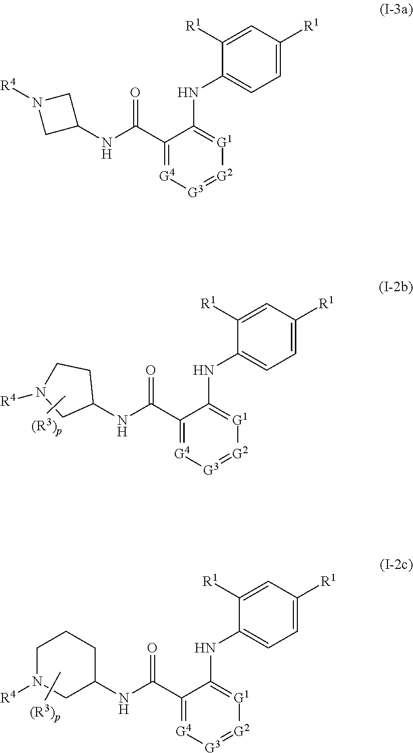
or a pharmaceutically acceptable salt, solvate or isotopic derivative thereof, wherein G¹, G², G³, G⁴, p, R¹, R², R³, and R⁴ are as defined for Formula (I).

4. The compound of claim 1, or a pharmaceutically acceptable salt, solvate or isotopic derivative thereof, wherein the compound of Formula (I) is a compound of Formula (I-3a), (I-3b), or (I-3c): 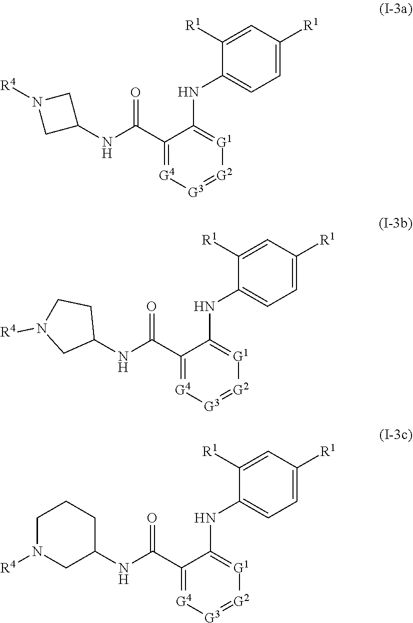
or a pharmaceutically acceptable salt, solvate or isotopic derivative thereof, wherein G¹, G², G³, G⁴, R¹, R², R³, and R⁴ are as defined for Formula (I).

5. The compound of any one of claims 1-4, or a pharmaceutically acceptable salt, solvate or isotopic derivative thereof, wherein G¹ is CH.

6. The compound of any one of claims 1-4, or a pharmaceutically acceptable salt, solvate or isotopic derivative thereof, wherein G¹ is CR².

7. The compound of any one of claims 1-5, or a pharmaceutically acceptable salt, solvate or isotopic derivative thereof, wherein G² is N.

8. The compound of any one of claims 1-5, or a pharmaceutically acceptable salt, solvate or isotopic derivative thereof, wherein G² is CH.

9. The compound of any one of claims 1-5, or a pharmaceutically acceptable salt, solvate or isotopic derivative thereof, wherein G² is CR².

10. The compound of any one of claims 1-8, or a pharmaceutically acceptable salt, solvate or isotopic derivative thereof, wherein G³ is N.

11. The compound of any one of claims 1-8, or a pharmaceutically acceptable salt, solvate or isotopic derivative thereof, wherein G³ is CH.

12. The compound of any one of claims 1-8, or a pharmaceutically acceptable salt, solvate or isotopic derivative thereof, wherein G³ is CR².

13. The compound of any one of claims 1-11, or a pharmaceutically acceptable salt, solvate or isotopic derivative thereof, wherein G⁴ is N.

14. The compound of any one of claims 1-11, or a pharmaceutically acceptable salt, solvate or isotopic derivative thereof, wherein G⁴ is CH.

15. The compound of any one of claims 1-11, or a pharmaceutically acceptable salt, solvate or isotopic derivative thereof, wherein G⁴ is CR².

16. The compound of any one of claims 1-14, or a pharmaceutically acceptable salt, solvate or isotopic derivative thereof, wherein G⁴ is CR².

17. The compound of any one of claims 1-4, or a pharmaceutically acceptable salt, solvate or isotopic derivative thereof, wherein G¹ is CH, G² is CF, G³ is CH, and G⁴ is CF.

18. The compound of any one of claims 1-4, or a pharmaceutically acceptable salt, solvate or isotopic derivative thereof, wherein wherein G¹ is CF, G² is CF, G³ is CF, and G⁴ is CF.

19. The compound of any one of claims 1-4, or a pharmaceutically acceptable salt, solvate or isotopic derivative thereof, wherein G¹ is CH, G² is N, G³ is CH, and G⁴ is CH.

20. The compound of any one of claims 1-4, or a pharmaceutically acceptable salt, solvate or isotopic derivative thereof, wherein G¹ is CH, G² is N, G³ is CH, and G⁴ is CF.

21. The compound of any one of claims 1-4, or a pharmaceutically acceptable salt, solvate or isotopic derivative thereof, wherein G¹ is CH, G² is N, G³ is CH, and G⁴ is CCl.

22. The compound of any one of claims 1-4, or a pharmaceutically acceptable salt, solvate or isotopic derivative thereof, wherein G¹ is CH, G² is CH, G³ is N, and G⁴ is CH.

23. The compound of any one of claims 1-22, or a pharmaceutically acceptable salt, solvate or isotopic derivative thereof, wherein each R¹ is independently selected from the group consisting of halogen and optionally substituted C₂-C₆ alkynyl.

24. The compound of any one of claims 1-22, or a pharmaceutically acceptable salt, solvate or isotopic derivative thereof, wherein m is 2 and each R¹ is independently selected from the group consisting of halogen and optionally substituted C₂-C₆ alkynyl.

25. The compound of claim 24, or a pharmaceutically acceptable salt, solvate or isotopic derivative thereof, wherein one R¹ is halogen and the other R¹ is optionally substituted C₂-C₆ alkynyl.

26. The compound of claim 25, or a pharmaceutically acceptable salt, solvate or isotopic derivative thereof, wherein one R¹ is fluoro and the other R¹ is ethynyl.

27. The compound of claim 24, or a pharmaceutically acceptable salt, solvate or isotopic derivative thereof, wherein each R¹ is indepdently selected from the group consisting of fluoro, chloro, bromo, and iodo.

28. The compound of claim 27, or a pharmaceutically acceptable salt, solvate or isotopic derivative thereof, wherein one R¹ is fluoro and the other R¹ is iodo.

29. The compound of claim 27, or a pharmaceutically acceptable salt, solvate or isotopic derivative thereof, wherein one R¹ is chloro and the other R¹ is bromo.

30. The compound of any one of claims 1-29, or a pharmaceutically acceptable salt, solvate or isotopic derivative thereof, wherein R⁴ is hydrogen.

31. The compound of any one of claims 1-29, or a pharmaceutically acceptable salt, solvate or isotopic derivative thereof, wherein R⁴ is selected from the group consisting of optionally substituted C₁-C₆ alkyl, optionally substituted C₁-C₆ haloalkyl, and -[(optionally substituted C₁-C₆ alkylene)-(optionally substituted C₁-C₆ alkoxy)].

32. The compound of claim 31, or a pharmaceutically acceptable salt, solvate or isotopic derivative thereof, wherein R⁴ is optionally substituted C₁-C₆ alkyl.

33. The compound of claim 32, or a pharmaceutically acceptable salt, solvate or isotopic derivative thereof, wherein R⁴ is selected from the group consisting of methyl, ethyl, and prop-2-yl.

34. The compound of claim 31, or a pharmaceutically acceptable salt, solvate or isotopic derivative thereof, wherein R⁴ is optionally substituted C₁-C₆ haloalkyl.

35. The compound of claim 34, or a pharmaceutically acceptable salt, solvate or isotopic derivative thereof, wherein R⁴ is 2-fluoroeth-1-yl.

36. The compound of claim 31, or a pharmaceutically acceptable salt, solvate or isotopic derivative thereof, wherein R⁴ is -[(optionally substituted C₁-C₆ alkylene)-(optionally substituted C₁-C₆ alkoxy)].

37. The compound of claim 36, or a pharmaceutically acceptable salt, solvate or isotopic derivative thereof, wherein R⁴ is 2-methoxyeth-1-yl.

38. A compound selected from a compound of Table 1:

| Compound | | |
|---|---|---|
| No. | Structure | Chemical Name |
| 1 | 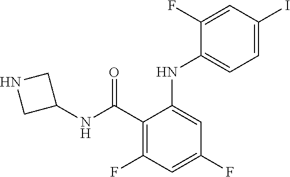 [SMILES: O=C(NC1CNC1)c1c(F)cc(F)cc1Nc1ccc(I)cc1F] | N-(azetidin-3-yl)-2,4- difluoro-6-((2-fluoro-4- iodophenyl)amino)benzamide |
| 2 | 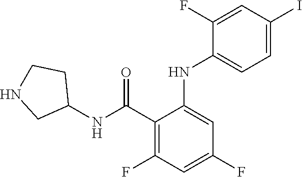 [SMILES: O=C(NC1CCNC1)c1c(F)cc(F)cc1Nc1ccc(I)cc1F] | 2,4-difluoro-6-((2-fluoro-4- iodophenyl)amino)-N- (pyrrolidin-3-yl)benzamide |
| 3 | 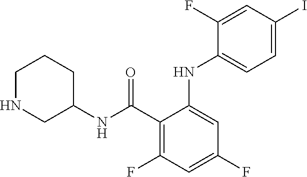 [SMILES: O=C(NC1CCCNC1)c1c(F)cc(F)cc1Nc1ccc(I)cc1F] | 2,4-difluoro-6-((2-fluoro-4- iodophenyl)amino)-N- (piperidin-3-yl)benzamide |
| 4 | 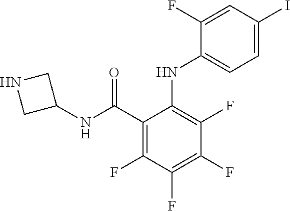 [SMILES: O=C(NC1CNC1)c1c(F)c(F)c(F)c(F)c1Nc1ccc(I)cc1F] | N-(azetidin-3-y1)-2,3,4,5- tetrafluoro-6-((2-fluoro-4- iodophenyl)amino)benzamide |
| 5 | 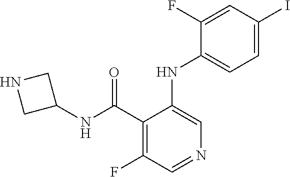 [SMILES: O=C(NC1CNC1)c1c(F)cncc1Nc1ccc(I)cc1F] | N-(azetidin-3-y1)-3-fluoro-5- ((2-fluoro-4-iodophenyl) amino)isonicotinamide |
| 6 | 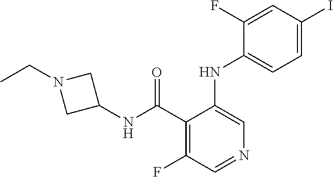 [SMILES: CCN1CC(NC(=O)c2c(F)cncc2Nc2ccc(I)cc2F)C1] | N-(1-ethylazetidin-3-y1)-3- fluoro-5-((2-fluoro-4- iodophenyl)amino) isonicotinamide |
| 7 | 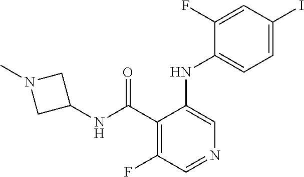 [SMILES: CN1CC(NC(=O)c2c(F)cncc2Nc2ccc(I)cc2F)C1] | 3-fluoro-5-((2-fluoro-4- iodophenyl)amino)-N-(1- methylazetidin-3- yl)isonicotinamide |
| 8 | 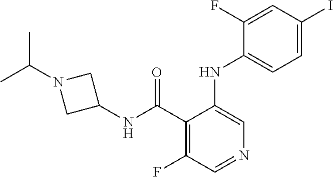 [SMILES: CC(C)N1CC(NC(=O)c2c(F)cncc2Nc2ccc(I)cc2F)C1] | 3-fluoro-5-((2-fluoro-4- iodophenyl)amino)-N-(1- isopropylazetidin-3- yl)isonicotinamide |
| 9 | 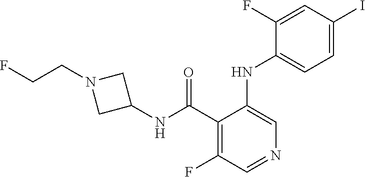 [SMILES: O=C(NC1CN(CCF)C1)c1c(F)cncc1Nc1ccc(I)cc1F] | 3-fluoro-5-((2-fluoro-4- iodophenyl)amino)-N-(1-(2- fluoroethyl)azetidin-3- yl)isonicotinamide |
| 10 | 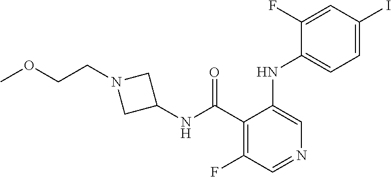 [SMILES: COCCN1CC(NC(=O)c2c(F)cncc2Nc2ccc(I)cc2F)C1] | 3-fluoro-5-((2-fluoro-4- iodophenyl)amino)-N-(1-(2- methoxyethyl)azetidin-3- yl)isonicotinamide |
| 11 | 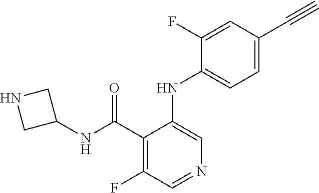 [SMILES: C#Cc1ccc(Nc2cncc(F)c2C(=O)NC2CNC2)c(F)c1] | N-(azetidin-3-y1)-3-((4- ethyny1-2- fluorophenyl)amino)-5- fluoroisonicotinamide |
| 12 | 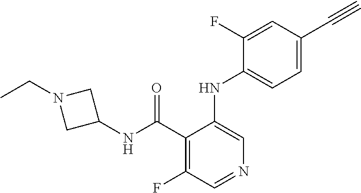 [SMILES: C#Cc1ccc(Nc2cncc(F)c2C(=O)NC2CN(CC)C2)c(F)c1] | N-(1-ethylazetidin-3-y1)-3- ((4-ethynyl-2- fluorophenyl)amino)-5- fluoroisonicotinamide |
| 13 | 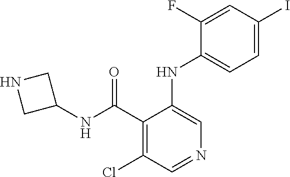 [SMILES: O=C(NC1CNC1)c1c(Cl)cncc1Nc1ccc(I)cc1F] | N-(azetidin-3-y1)-3-chloro-5- ((2-fluoro-4-iodophenyl) amino)isonicotinamide |
| 14 | 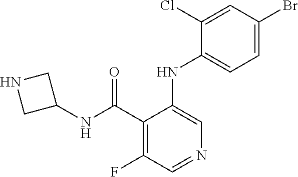 [SMILES: O=C(NC1CNC1)c1c(F)cncc1Nc1ccc(Br)cc1Cl] | N-(azetidin-3-yl)-3-((4- bromo-2- chlorophenyl)amino)-5- fluoroisonicotinamide |
| 15 | 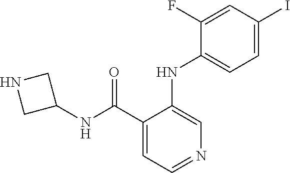 [SMILES: O=C(NC1CNC1)c1ccncc1Nc1ccc(I)cc1F] | N-(azetidin-3-yl)-3-((2- fluoro-4- iodophenyl)amino) isonicotinamide |
| 16 | 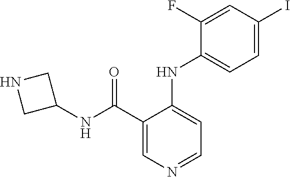 [SMILES: O=C(NC1CNC1)c1cnccc1Nc1ccc(I)cc1F] | N-(azetidin-3-yl)-4-((2- fluoro-4- iodophenyl)amino) nicotinamide |
| 17 | 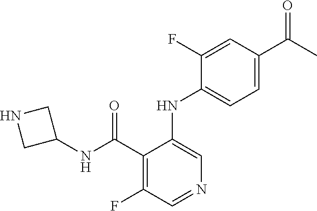 [SMILES: CC(=O)c1ccc(Nc2cncc(F)c2C(=O)NC2CNC2)c(F)c1] | 3-((4-acetyl-2- fluorophenyl)amino)-N- (azetidin-3-y1)-5- fluoroisonicotinamide |
| 18 | 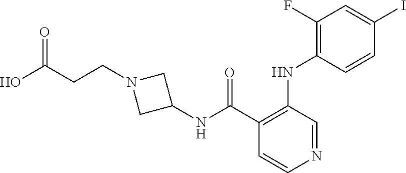 [SMILES: O=C(O)CCN1CC(NC(=O)c2ccncc2Nc2ccc(I)cc2F)C1] | 3-(3-(3-((2-fluoro-4- iodophenyl)amino)isonicotina mido)azetidin-1-yl)propanoic acid |
| 19 | 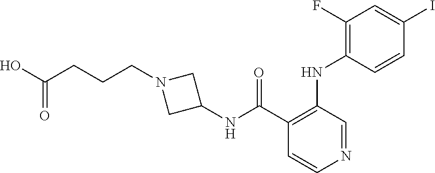 [SMILES: O=C(O)CCCN1CC(NC(=O)c2ccncc2Nc2ccc(I)cc2F)C1] | 4-(3-(3-((2-fluoro-4- iodophenyl)amino)isonicotina mido)azetidin-1-yl)butanoic acid |
| 20 | 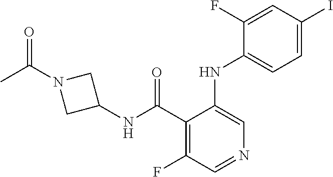 [SMILES: CC(=O)N1CC(NC(=O)c2c(F)cncc2Nc2ccc(I)cc2F)C1] | N-(1-acetylazetidin-3-y1)-3- fluoro-5-((2-fluoro-4- iodophenyl)amino) isonicotinamide |

or a pharmaceutically acceptable salt, solvate or isotopic derivative thereof.

39. A pharmaceutical composition comprising a compound of any one of claims 1-38, or a pharmaceutically acceptable salt thereof, and a pharmaceutically acceptable diluent or carrier.

40. A combination comprising a compound of any of claims 1-38, or a pharmaceutically acceptable salt thereof, and a second prophylactic or therapeutic agent.

41. A compound according to any one of claims 1-38 for use in treating and/or preventing a proliferation disorder, such as a cancer, or a tumor in a subject.

42. The compound of claim 41, wherein the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

43. A method for treating and/or preventing a proliferation disorder, such as a cancer, or a tumor in a subject, wherein the method comprises administering to the subject an effective amount of a compound of any one of claims 1-38, or a pharmaceutically acceptable salt thereof, a pharmaceutical composition of claim 39, or a combination of claim 40.

44. The method of claim 43, wherein the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

45. Use of a compound according to any of claims 1-38 for the manufacture of a medicament.

46. A method for inhibiting an activity of one or more kinases selected from the group consisting of MEK, COT1, FGFR4, MINK, MYO3A, PKG1B, and PLK3, comprising contacting the cell with an effective amount of a compound of any one of claims 1-38, or a pharmaceutically acceptable salt thereof, a pharmaceutical composition of claim 39, or a combination of claim 40, wherein the contacting is in vitro, ex vivo, or in vivo.

47. A compound according to any one of claims 1-38 for use in the treatment of a neurodegenerative disease.

48. The compound of claim 47, wherein the neurodegenerative disease is selected from the group consisting of Amyotrophic lateral sclerosis, Parkinson's disease, Alzheimer's disease, and Huntington's disease.

49. A method for treating a neurodegenerative disease in a subject, wherein the method comprises administering to the subject an effective amount of a compound of any one of claims 1-38, or a pharmaceutically acceptable salt thereof, a pharmaceutical composition of claim 39, or a combination of claim 40.

50. The method of claim 49, wherein the neurodegenerative disease is selected from the group consist of Amyotrophic lateral sclerosis, Parkinson's disease, Alzheimer's disease, and Huntington's disease.

51. A method for treating an immunodeficient disease in a subject, wherein the method comprises administering to the subject an effective amount of a compound of any one of claims 1-38, or a pharmaceutically acceptable salt thereof, a pharmaceutical composition of claim 39, or a combination of claim 40.

52. The method of claim 51, wherein the neurodegenerative disease is selected from the group consist of immunodeficient disease is selected from the group consist of cancers, infectious disease, and some genetic diseases.

53. A method for inhibiting an activity of one or more kinases selected from the group consisting of MEK, COT1, FGFR4, MINK, MYO3A, PKG1B, and PLK3, comprising contacting the cell with an effective amount of a compound of any one of claims 1-38, or a pharmaceutically acceptable salt thereof, a pharmaceutical composition of claim 39, or a combination of claim 40, wherein the contacting is in vitro, ex vivo, or in vivo.
